(19) United States
(12) Patent Application Publication
(10) Pub. No.: US 2016/0356790 A1
(43) Pub. Date: Dec. 8, 2016

(54) SIGNALING PATHWAYS IN TISSUES FROM INFLAMMATORY BOWEL DISEASE PATIENTS

(71) Applicant: NESTEC S.A., Vevey (CH)

(72) Inventors: SHARAT SINGH, Rancho Santa Fe, CA (US); KATHERINE DRAKE, San Diego, CA (US); ANJALI JAIN, San Diego, CA (US); RICHARD KIRKLAND, San Diego, CA (US); XINJUN LIU, San Diego, CA (US)

(73) Assignee: NESTEC S.A., Vevey (CH)

(21) Appl. No.: 15/171,970

(22) Filed: Jun. 2, 2016

Related U.S. Application Data
(63) Continuation of application No. PCT/IB2014/066529, filed on Dec. 2, 2014.
(60) Provisional application No. 61/987,358, filed on May 1, 2014.
(60) Provisional application No. 61/911,428, filed on Dec. 3, 2013.

(51) Int. Cl. G01N 33/68; C12Q 1/68

(52) U.S. Cl. CPC G01N 33/6893; C12Q 1/6883; C12Q 2600/106; C12Q 2600/158; G01N 2800/52; G01N 2800/065; G01N 2333/71; G01N 2333/91205; G01N 2333/65; G01N 2333/525

(57) ABSTRACT

The present invention provides methods for measuring the total and/or activation levels of one or more analytes such as HER1, HER2, HER3, cMET, IGF-1R, PI3K, AKT, PRAS40, MEK, RSK, JAK1, STAT1, STAT3, TNFα, and/or anti-TNFα drug to determine whether a subject with inflammatory bowel disease (IBD) has non-inflamed and/or non-involved or inflamed and/or involved gastrointestinal tissue. The methods of the present invention can be used to evaluate mucosal healing, to diagnose, prognose, and monitor the progression of IBD, and to select and monitor the therapeutic response to IBD therapy.

### CROSS-REFERENCE TO RELATED APPLICATIONS

This application is a continuation of PCT/IB2014/066529 filed Dec. 2, 2014, which application claims priority to U.S. Provisional Application No. 61/911,428, filed Dec. 3, 2013, and U.S. Provisional Application No. 61/987,358, filed May 1, 2014, the disclosures of which are hereby incorporated by reference in their entirety for all purposes.

### BACKGROUND OF THE INVENTION

Inflammatory bowel disease (IBD) consists of Crohn's Disease (CD) and ulcerative colitis (UC). In both diseases, the bowel of the subject is inflamed. Endoscopic mucosal healing correlates with clinical outcomes and has been proposed as a goal for targeted therapeutics. Moreover, mucosal healing is a key prognostic factor in the management of IBD. However, very little is known about the specific molecular pathways that may be contributing to the mucosal healing process, nor is the identification of biomarkers indicative of mucosal healing known.

Abnormal signaling pathways play a role in the dysregulation of the inflammatory response. By measuring certain signal transduction analytes in inflammatory pathways, it was believed that these analytes could be indicators of mucosal healing. These data could then be used for identification of optimum therapeutics or as an indication as to whether targeted therapy was efficacious.

In view of the foregoing, the present invention provides methods to evaluate inflamed, non-inflamed, involved, or non-involved gastrointestinal tissue to diagnose IBD. The results from these methods can in turn be used to evaluate therapy, prognostic outcomes, and mucosal healing.

### BRIEF SUMMARY OF THE INVENTION

In some aspects, the present invention provides a method for determining whether a subject having inflammatory bowel disease (IBD) has non-inflamed and/or non-involved or inflamed and/or involved gastrointestinal tissue, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) comparing the total level and/or activation level of the one or more analytes measured in step (a) to that of a control, thereby determining whether the subject has non-inflamed and/or non-involved or inflamed and/or involved gastrointestinal tissue.

In other aspects, the present invention provides a method for selecting an optimal IBD therapy for the treatment of a subject having IBD, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) selecting an optimal IBD therapy for the subject having IBD based upon the total level and/or activation level calculated in step (a) compared to that of a control.

In yet other aspects, the present invention provides a method for monitoring the therapeutic response to IBD therapy in a subject having IBD, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) determining whether the subject is responding to the IBD therapy based upon the total expression level and/or activation level of one or more analytes measured in step (a) compared to that of a control.

In further aspects, the present invention provides a method for diagnosing early onset inflammatory bowel disease (IBD) in a subject, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) comparing the total level and/or activation level of the one or more analytes measured in step (a) to that of a control, thereby determining that the subject has early onset IBD if the total level and/or activation level of the one or more analytes is higher compared to that of the control.

In other aspects, the present invention provides a method for diagnosing ulcerative colitis (UC) in a subject, the method comprising:
(a) measuring the total level and/or activation level of STAT3 in a colon cell lysate, wherein the colon cell lysate is produced by lysing a cell from a colon sample taken from the subject;
(b) measuring the total level and/or activation level of STAT3 in an ileum cell lysate, wherein the ileum cell lysate is produced by lysing a cell from an ileum sample taken from the subject; and
(c) comparing the total level and/or activation level of STAT3 in the colon cell lysate to that of the ileum cell lysate, thereby diagnosing UC in the subject.

Other objects, features, and advantages of the present invention will be apparent to one of skill in the art from the following detailed description and figures.

### BRIEF DESCRIPTION OF THE DRAWINGS

FIGS. 1A-1C illustrate the total expression of growth factor driven signaling molecules such as HER1 (FIG. 1A), cMET (FIG. 1B) and HER2 (FIG. 1C) in inflamed/involved tissues as compared to the non-inflamed/non-involved tissues from individuals having IBD (e.g., CD or UC). The asterisk denotes statistical significance.

FIGS. 2A-2E illustrate the level of activated (e.g., phosphorylated) over total expression of growth factor driven signaling molecules such as HER1 (FIG. 2A), HER2 (FIG. 2B), HER2 (FIG. 2C), PI3K (FIG. 2D), and IGF-1R (FIG. 2E) in inflamed/involved tissue versus the non-inflamed/non-involved tissue from individuals having IBD (e.g., CD or UC). The asterisk denotes statistical significance. For instance, FIG. 2C shows the level of activated HER3 divided by the level of total expression of HER3 in inflamed/involved tissue vs. non-inflamed/non-involved tissue.

FIGS. 3A-3E illustrate the level of activated (e.g., phosphorylated) growth factor driven signaling molecules such as activated HER1 (FIG. 3A), activated HER2 (FIG. 3B), activated HER2 (FIG. 3C), and activated IGF-1R (FIG. 3E) in non-inflamed/non-involved colonic tissue versus non-inflamed/non-involved ileal tissue from individuals having IBD. Total cMET (FIG. 3D) levels were measured and analyzed. The asterisk denotes statistical significance.

FIGS. 4A-4H illustrate the total expression level and/or the activated (e.g., phosphorylated) level of analytes (e.g., JAK-STAT signaling molecules, PI3K-AKT-PRAS40 signaling molecules, and MEK-RSK signaling molecules) such as activated PI3K (FIG. 4A), activated AKT (FIG. 4B), activated JAK1 (FIG. 4), activated RSK (FIG. 4D), activated MEK (FIG. 4E), activated STAT3 (FIG. 4F), activated STAT1 (FIG. 4G) and activated PRAS40 (FIG. 4H) in non-inflamed/non-involved colonic tissue versus non-inflamed/non-involved ileal tissue from individuals having IBD (e.g., CD or UC). The asterisk denotes statistical significance.

FIGS. 5A-5F illustrate the expression levels of several epithelial cell analytes such as HER1 (FIG. 5A), HER2 (FIG. 5B), HER3 (FIG. 5C), cMET (FIG. 5D) and IGF-1R (FIG. 5E) in non-inflamed tissue of the colon vs. ileum in individuals with IBD (e.g., CD or UC). The levels of HER1 (FIG. 5A), HER3 (FIG. 5C) and cMET (FIG. 5D) levels were statistically significantly higher in colonic tissue vs. ileal tissue. The level of the reference (control) analyte, e.g., CK, was not different between the colonic and ileal tissues (FIG. 5F). The asterisk denotes statistical significance.

FIG. 6 illustrates that epithelial cell markers are higher in non-inflamed, non-involved IBD tissues than in inflamed or involved tissues.

FIG. 7 illustrates that several epithelial cell markers are also significantly higher in the colon than in the ileum.

FIGS. 8A-B illustrate that growth factor and cytokine driven, activated signaling pathways are expressed to higher levels in the colon vs. the ileum.

FIG. 9 illustrates that HER2 normalized TNFα expression and Drug (anti-TNFα) are significantly higher in inflamed IBD tissues as compared to non-inflamed tissues.

### DETAILED DESCRIPTION OF THE INVENTION

### I. Introduction

The present invention provides methods for measuring the total (e.g., expression) and/or activation (e.g., phosphorylation) levels of one or more analytes such as HER1, HER2, HER3, cMET, IGF-1R, PI3K, AKT, PRAS40, MEK, RSK, JAK1, STAT1, STAT3, TNFα, anti-TNFα drug, and a combination thereof, to determine whether a subject has non-inflamed and/or non-involved or inflamed and/or involved gastrointestinal tissue and/or is undergoing or has undergone mucosal healing. In addition, the present invention provides methods for diagnosing early onset inflammatory bowel disease. Further provided are methods for diagnosing ulcerative colitis in a subject. The present invention also provides methods for selecting therapy for the treatment of IBD, as well as methods for monitoring a subject's therapeutic response to an anti-TNFα agent or a combination therapy comprising an anti-TNFα agent.

### II. Definitions

As used herein, the following terms have the meanings ascribed to them unless specified otherwise.

The term “inflamed gastrointestinal tissue” typically refers to a tissue of the gastrointestinal tract that has inflammation. For instance, an individual with ulcerative colitis may have inflammation of the colon. An individual with Crohn's disease may exhibit segmental transmural inflammatory lesions including fistulas and abscesses anywhere along the gastrointestinal tract.

The term “involved gastrointestinal tissue” typically refers to a tissue of the gastrointestinal tract that has inflammation and is involved in disease, or that had previous inflammation and was previously involved in disease (e.g., the subject's gastrointestinal tissue had overt disease in previous examinations, but there was no evidence of disease activity at the time of sampling). For example, a tissue is determined to be involved or previously involved if the tissue has been involved in IBD and/or has had scarring, but was not inflamed at the time of sampling, observation, or analysis.

The term “non-inflamed gastrointestinal tissue” typically refers to a tissue of the gastrointestinal tract that has no evidence of inflammation due to IBD.

The term “non-involved gastrointestinal tissue” typically refers to a tissue of the gastrointestinal tract that has no evidence of involvement in IBD.

The term “aggressive IBD” refers to a disease state of inflammatory bowel disease wherein the patient has, for example, a high relapse rate, a need for clinical admission and/or surgery, developed colon cancer, has extraintestinal manifestations, developed penetrating disease, a need for repeat surgery, multiple clinical admissions due to flares or a combination thereof. Aggressive Crohn's disease can include involvement of the upper gastrointestinal tract and ileum, penetrating disease, early age of diagnosis, smoking, extra ulcerations of the gastrointestinal mucosa, high titers of serum antibodies (including autoantibodies), and mutations in genetic markers such as NOD2. Aggressive ulcerative colitis can include involvement of the colon, a higher risk of needing a colectomy, a higher risk of developing colon cancer, plasma cell infiltration of the colonic mucosa, and crypt atrophy. See, e.g., Yarur et al., Gastroenterol. Hepatol. (N.Y.), 7(10):652-659 (2011).

The term “less aggressive IBD” refers to a disease state of inflammatory bowel disease wherein the patient exhibits symptoms or a conditions that is less severe than aggressive IBD.

The term “mucosal healing” refers to restoration of normal mucosal appearance of a previously inflamed region, and complete absence of ulceration and inflammation at the endoscopic and microscopic levels. Mucosal healing includes repair and restoration of the mucosa, submucosa, and muscularis layers. It can also include neuronal and lymphangiogenic elements of the intestinal wall.

The term “progression of mucosal healing” refers to a transition through the phases (e.g., stages) of mucosal healing from inflammatory phase, proliferation phase and remodeling phase towards complete improvement (e.g., complete repair) of the intestinal mucosa.

The term “subject,” “patient,” or “individual” typically refers to humans, but also to other animals including, e.g., other primates, rodents, canines, felines, equines, ovines, porcines, and the like.

The term “sample” as used herein includes any biological specimen obtained from a patient. Samples include, without limitation, a tissue sample such as a biopsy of a site of inflammation or non-inflammation (e.g., needle biopsy), whole blood, plasma, serum, red blood cells, white blood cells (e.g., peripheral blood mononuclear cells (PBMC), polymorphonuclear (PMN) cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration), any other bodily fluid, and cellular extracts thereof. In some embodiments, the sample is a tissue biopsy, e.g., tissue obtained from a site of inflammation or non-inflammation such as a portion of the gastrointestinal tract or synovial tissue. In some instances, the tissue biopsy is taken from a particular segment of the gastrointestinal tract such as the esophagus, stomach, duodenum, duodenum, jejunum, ileum, cecum, colon, rectum, anus and ligament of Trietz. In other embodiments, the sample is whole blood or a fractional component thereof such as plasma, serum, or a cell pellet. In other embodiments, the sample is obtained by isolating PBMCs and/or PMN cells using any technique known in the art. In yet other embodiments, the sample is a formalin fixed paraffin embedded (FFPE) tissue sample, e.g., from a colon tissue sample or an ileum tissue sample. In particular embodiments, the sample is a tissue lysate or extract prepared from frozen tissue obtained from a subject having IBD. In preferred embodiments, the sample is a cell lysate from a tissue sample obtained from a biopsy, e.g., a fine needle aspiration biopsy. In other embodiments, the sample is a cell lysate from a tissue sample obtained from an endoscopic procedure.

As used herein, “EUS/FNA” includes endoscopic ultrasound (EUS) and fine needle aspiration, wherein EUS is a technique using ultrasound during an endoscopic procedure to look at or through the wall of the gastrointestinal tract. This technique allows physicians to see organs and structures not typically visible during gastrointestinal endoscopy, such as the layers of the gastrointestinal tract wall, the liver, pancreas, lymph nodes, and bile ducts. The scope used for EUS is similar to a regular endoscope with the added component of an ultrasound transducer. Under continuous real-time ultrasound guidance, a thin needle can be advanced into these structures to obtain an aspirate of the tissue. This technique is known as a fine needle aspirate (FNA).

The term “analyte” include any signal transduction molecule, biochemical markers, serological markers, protein markers, therapeutic drug, and/or other clinical or echographic characteristics, that can be measured in a sample. In certain embodiments, an analyte of the invention can be used to detect inflamed and/or involved tissue in a sample from an individual with a disease such as IBD including Crohn's disease and ulcerative colitis. The term also includes any molecule of interest, typically a macromolecule such as a polypeptide, whose presence, amount (expression level), activation state (e.g., phosphorylation, glycosylation, sumoylation, ubiquination, nitrosylation, methylation, acetylation, lipidation and the like) and/or identity is determined.

The term “signal transduction molecule” or “signal transducer” includes proteins and other molecules that carry out the process by which a cell converts an extracellular signal or stimulus into a response, typically involving ordered sequences of biochemical reactions inside the cell. Examples of signal transduction molecules include, but are not limited to, receptor tyrosine kinases such as EGFR (e.g., EGFR/HER1/ErbB1, HER2/Neu/ErbB2, HER3/ErbB3, HER4/ErbB4), VEGFR1/FLT1, VEGFR2/FLK1/KDR, VEGFR3/FLT4, FLT3/FLK2, PDGFR (e.g., PDGFRA, PDGFRB), c-KIT/SCFR, INSR (insulin receptor), IGF-IR, IGF-IIR, IRR (insulin receptor-related receptor), CSF-1R, FGFR 1-4, HGFR 1-2, CCK4, TRK A-C, cMET, RON, EPHA 1-8, EPHB 1-6, AXL, MER, TYRO3, TIE 1-2, TEK, RYK, DDR 1-2, RET, c-ROS, V-cadherin, LTK (leukocyte tyrosine kinase), ALK (anaplastic lymphoma kinase), ROR 1-2, MUSK, AATYK 1-3, and RTK 106; truncated forms of receptor tyrosine kinases such as truncated HER2 receptors with missing amino-terminal extracellular domains (e.g., p95ErbB2 (p95m), p110, p95c, p95n, etc.), truncated cMET receptors with missing amino-terminal extracellular domains, and truncated HER3 receptors with missing amino-terminal extracellular domains; receptor tyrosine kinase dimers (e.g., p95HER2/HER3; p95HER2/HER2; truncated HER3 receptor with HER1, HER2, HER3, or HER4; HER2/HER2; HER3/HER3; HER2/HER3; HER1/HER2; HER1/HER3; HER2/HER4; HER3/HER4; etc.); non-receptor tyrosine kinases such as BCR-ABL, Src, Frk, Btk, Csk, Abl, Zap70, Fes/Fps, Fak, JAK (e.g., JAK1, JAK2, JAK3, TYK2), Ack, and LIMK; tyrosine kinase signaling cascade components such as AKT (e.g., AKT1, AKT2, AKT3), MEK (MAP2K1), ERK2 (MAPK1), ERK1 (MAPK3), PI3K (e.g., PIK3CA (p110), PIK3R1 (p85)), PDK1, PDK2, phosphatase and tensin homolog (PTEN), SGK3, 4E-BP1, P70S6K (e.g., p70 S6 kinase splice variant alpha I), protein tyrosine phosphatases (e.g., PTP1B, PTPN13, BDP1, etc.), RAF, PLA2, MEKK, JNKK, JNK, p38, Shc (p66), Ras (e.g., K-Ras, N-Ras, H-Ras), Rho, Rac1, Cdc42, PLC, PKC, p53, cyclin D1, SHP-1, SHP-2, SHP-3, SOCS, SOCS3, STAM, STAT1, STAT3, STATS, PIAS, PTP, phosphatidylinositol 4,5-bisphosphate (PIP2), phosphatidylinositol 3,4,5-trisphosphate (PIP3), phosphatidylinositol 4-phosphate 5-kinase (PIP5K), mTOR, mTORC1, mTORC2 (e.g., GβL, Rictor, Sin1, PRR5/Protor-1, Deptor), Raptor, GβL (MLST8), Deptor, Rag (Rag A-D), Ragulator (LAMTOR1-5), BAD, p21, p2′7, ROCK, IP3, TSP-1, NOS, GSK-3β, RSK 1-3, JNK, c-Jun, Rb, CREB, Ki67, paxillin, 4E-BP1, 14-3-3, AMPKB-RAF, Bim, BRF1, CREB, FKBP12, IRS1, MKK4, MLK3, MST1, MST2, NDRG2, PLCG1, PKCα, PPP1CA, PRAS40, PRPK, QIK, RANBP3, REDD1/2, p70 S6 kinase, SGK1, SH3BP4, SH3RF1, SRPK2, STXBP4, TSC1, TSC2, TTC3, ULK1, ATG1, ATG13, USPS, and VCP; nuclear hormone receptors such as estrogen receptor (ER), progesterone receptor (PR), androgen receptor, glucocorticoid receptor, mineralocorticoid receptor, vitamin A receptor, vitamin D receptor, retinoic acid receptor, retinoid receptor, thyroid hormone receptor, and orphan receptors; nuclear receptor coactivators and repressors such as amplified in breast cancer-1 (AIB1) and nuclear receptor corepressor 1 (NCOR), respectively; and combinations thereof.

The term “activation state” refers to whether a particular signal transduction molecule is activated. Similarly, the term “activation level” refers to what extent a particular signal transduction molecule is activated. The activation state typically corresponds to the phosphorylation, ubiquitination, and/or complexation status of one or more signal transduction molecules. Non-limiting examples of activation states (listed in parentheses) include: HER1/EGFR (EGFRvIII, phosphorylated (p-) EGFR, EGFR: Shc, ubiquitinated (u-) EGFR, p-EGFRvIII); HER2/ErbB2 (p-ErbB2, p95HER2 (truncated ErbB2), p-p95HER2, ErbB2:Shc, ErbB2:PI3K, ErbB2:EGFR, ErbB2:ErbB3, ErbB2:ErbB4); HER3/ErbB3 (p-ErbB3, truncated ErbB3, ErbB3:PI3K, p-ErbB3:PI3K, ErbB3:Shc); HER4/ErbB4 (p-ErbB4, ErbB4: Shc); c-MET (p-c-MET, truncated c-MET, c-Met:HGF complex); AKT1 (p-AKT1); AKT2 (p-AKT2); AKT3 (p-AKT3); PTEN (p-PTEN); P70S6K (p-P70S6K); MEK (p-MEK); ERK1 (p-ERK1); ERK2 (p-ERK2); PDK1 (p-PDK1); PDK2 (p-PDK2); SGK3 (p-SGK3); 4E-BP1 (p-4E-BP1); PIK3R1 (p-PIK3R1); c-KIT (p-c-KIT); ER (p-ER); IGF-1R (p-IGF-1R, IGF-1R:IRS, IRS:PI3K, p-IRS, IGF-1R:PI3K); INSR (p-INSR); FLT3 (p-FLT3); HGFR1 (p-HGFR1); HGFR2 (p-HGFR2); RET (p-RET); PDGFRA (p-PDGFRA); PDGFRB (p-PDGFRB); VEGFR1 (p-VEGFR1, VEGFR1:PLCγ, VEGFR1:Src); VEGFR2 (p-VEGFR2, VEGFR2:PLCγ, VEGFR2:Src, VEGFR2:heparin sulphate, VEGFR2:VE-cadherin); VEGFR3 (p-VEGFR3); FGFR1 (p-FGFR1); FGFR2 (p-FGFR2); FGFR3 (p-FGFR3); FGFR4 (p-FGFR4); TIE1 (p-TIE1); TIE2 (p-TIE2); EPHA (p-EPHA); EPHB (p-EPHB); GSK-3β (p-GSK-3β); NF-KB (p-NF-KB), IKB (p-IKB, p-P65:IKB); BAD (p-BAD, BAD:14-3-3); PRAS40 (p-PRAS40), mTOR (p-mTOR); Rsk-1 (p-Rsk-1); Jnk (p-Jnk); P38 (p-P38); PI3K (p-PI3K), JAK1 (p-JAK1); JAK2 (p-JAK2); JAK3 (p-JAK3); TYK2 (p-TYK2); STAT1 (p-STAT1); STAT3 (p-STAT3); STATS (p-STATS); FAK (p-FAK); RB (p-RB); Ki67; MEK (p-MEK); p53 (p-p53); RSK (p-RSK); CREB (p-CREB); c-Jun (p-c-Jun); c-Src (p-c-Src); paxillin (p-paxillin); GRB2 (p-GRB2), Shc (p-Shc), Ras (p-Ras), GAB1 (p-GAB1), SHP2 (p-SHP2), GRB2 (p-GRB2), CRKL (p-CRKL), PLCγ (p-PLCγ), PKC (e.g., p-PKCα, p-PKCβ, p-PKCδ), adducin (p-adducin), RB1 (p-RB1), and PYK2 (p-PYK2).

The term “TNFα” is intended to include a human cytokine that exists as a 17 kDa secreted form and a 26 kDa membrane associated form, the biologically active form of which is composed of a trimer of noncovalently bound 17 kDa molecules. The structure of TNFα is described further in, for example, Jones et al., Nature, 338:225-228 (1989). The term TNFα is intended to include human TNFα, a recombinant human TNFα (rhTNF-α), or TNFα that is at least about 80% identity to the human TNFα protein. Human TNFα consists of a 35 amino acid (aa) cytoplasmic domain, a 21 aa transmembrane segment, and a 177 aa extracellular domain (ECD) (Pennica, D. et al. (1984) Nature 312:724). Within the ECD, human TNFα shares 97% aa sequence identity with rhesus TNFα, and 71% to 92% aa sequence identity with bovine, canine, cotton rat, equine, feline, mouse, porcine, and rat TNFα. TNFα can be prepared by standard recombinant expression methods or purchased commercially (R & D Systems, Catalog No. 210-TA, Minneapolis, Minn.).

In certain embodiments, “TNFα” is an “antigen,” which includes a molecule or a portion of the molecule capable of being bound by an anti-TNF-α drug. TNFα can have one or more than one epitope. In certain instances, TNFα will react, in a highly selective manner, with an anti-TNFα antibody. Preferred antigens that bind antibodies, fragments, and regions of anti-TNFα antibodies include at least 5 amino acids of human TNFα. In certain instances, TNFα is a sufficient length having an epitope of TNFα that is capable of binding anti-TNFα antibodies, fragments, and regions thereof.

An “array” or “microarray” comprises a distinct set and/or dilution series of capture antibodies immobilized or restrained on a solid support such as, for example, glass (e.g., a glass slide), plastic, chips, pins, filters, beads (e.g., magnetic beads, polystyrene beads, etc.), paper, membrane (e.g., nylon, nitrocellulose, polyvinylidene fluoride (PVDF), etc.), fiber bundles, or any other suitable substrate. The capture antibodies are generally immobilized or restrained on the solid support via covalent or noncovalent interactions (e.g., ionic bonds, hydrophobic interactions, hydrogen bonds, Van der Waals forces, dipole-dipole bonds). In certain instances, the capture antibodies comprise capture tags which interact with capture agents bound to the solid support. The arrays used in the assays described herein typically comprise a plurality of different capture antibodies and/or capture antibody concentrations that are coupled to the surface of a solid support in different known/addressable locations.

The term “capture antibody” is intended to include an immobilized antibody which is specific for (i.e., binds, is bound by, or forms a complex with) one or more analytes of interest in a sample such as a cellular extract. In particular embodiments, the capture antibody is restrained on a solid support in an array. Suitable capture antibodies for immobilizing any of a variety of signal transduction molecules on a solid support are available from Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.).

The term “detection antibody” as used herein includes an antibody comprising a detectable label which is specific for (i.e., binds, is bound by, or forms a complex with) one or more analytes of interest in a sample. The term also encompasses an antibody which is specific for one or more analytes of interest, wherein the antibody can be bound by another species that comprises a detectable label. Examples of detectable labels include, but are not limited to, biotin/streptavidin labels, nucleic acid (e.g., oligonucleotide) labels, chemically reactive labels, fluorescent labels, enzyme labels, radioactive labels, and combinations thereof. Suitable detection antibodies for detecting the activation state and/or total amount of any of a variety of signal transduction molecules are available from Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.). As a non-limiting example, phospho-specific antibodies against various phosphorylated forms of signal transduction molecules such as HER1, HER2, HER3, IGF1R, PI3K, AKT, ERK, RSK, cMET, IGF1R, JAK1, STAT1, STAT3, PRAS40, RSK, RPS6, c-KIT, c-Src, FLK-1, PDGFRA, PDGFRB, MAPK, PTEN, Raf, and MEK are available from Santa Cruz Biotechnology.

The term “activation state-dependent antibody” includes a detection antibody which is specific for (i.e., binds, is bound by, or forms a complex with) a particular activation state of one or more analytes of interest in a sample. In preferred embodiments, the activation state-dependent antibody detects the phosphorylation, ubiquitination, and/or complexation state of one or more analytes such as one or more signal transduction molecules. In some embodiments, the phosphorylation of members of the EGFR family of receptor tyrosine kinases and/or the formation of heterodimeric complexes between EGFR family members is detected using activation state-dependent antibodies. In particular embodiments, activation state-dependent antibodies are useful for detecting one or more sites of phosphorylation in one or more of the following signal transduction molecules (phosphorylation sites correspond to the position of the amino acid in the human protein sequence): EGFR/HER1/ErbB1 (e.g., tyrosine (Y) 1068); ErbB2/HER2 (e.g., Y1248); ErbB3/HER3 (e.g., Y1289); ErbB4/HER4 (e.g., Y1284); c-Met (e.g., Y1003, Y1230, Y1234, Y1235, and/or Y1349); IGFR1 (e.g., Y1158, Y1161, Y1162 and/or Y1163); PI3K (e.g., Y199, Y458, Y467, and/or Y688); JAK1 (e.g., Y3, 5216, Y217, Y220, S228, 5333, Y568, Y1022, Y1023); MEK (e.g., S217 and/or S221); AKT1 (e.g., S473 and/or T308); AKT2 (e.g., S474 and/or T309); AKT3 (e.g., S472 and/or T305); STAT1 (e.g., Y701, and/or S727); STAT3 (e.g., Y705 and/or S727); Rsk-1 (e.g., T357, T359, T573, 5221, 5380 and/or S363); PRAS40 (e.g., S183, 5221 and/or T246); CK (e.g., S23, S73, and/or S431); SGK3 (e.g., T256 and/or S422); 4E-BP1 (e.g., T70); ERK1 (e.g., T185, Y187, T202, and/or Y204); ERK2 (e.g., T185, Y187, T202, and/or Y204); PIK3R1 (e.g., Y688); PDK1 (e.g., S241); P70S6K (e.g., T229, T389, and/or S421); PTEN (e.g., S380); GSK-3β (e.g., S9); NF-KB (e.g., S536); IKB (e.g., S32); BAD (e.g., S112 and/or S136); mTOR (e.g., S2448); JNK (e.g., T183 and/or Y185); P38 (e.g., T180 and/or Y182); SHC (e.g., Y239, Y240, Y317, Y349, and/or Y427); FAK (e.g., Y397, Y576, 5722, Y861, and/or S910); RB (e.g., S249, T252, 5612, and/or S780); RB1 (e.g., S780); adducin (e.g., S662 and/or S724); PYK2 (e.g., Y402 and/or Y881); PKCα (e.g., S657); PKCα/β (e.g., T368 and/or T641); PKCδ (e.g., T505); p53 (e.g., S392 and/or S20); CREB (e.g., S133); c-Jun (e.g., S63); c-Src (e.g., Y416); and paxillin (e.g., Y31 and/or Y118).

The term “activation state-independent antibody” includes a detection antibody which is specific for (i.e., binds, is bound by, or forms a complex with) one or more analytes of interest in a sample irrespective of their activation state. For example, the activation state-independent antibody can detect both phosphorylated and unphosphorylated forms of one or more analytes such as one or more signal transduction molecules.

The term “incubating” is used synonymously with “contacting” and “exposing” and does not imply any specific time or temperature requirements unless otherwise indicated.

As used herein, an entity that is modified by the term “labeled” includes any entity, molecule, protein, enzyme, antibody, antibody fragment, cytokine, or related species that is conjugated with another molecule or chemical entity that is empirically detectable. Chemical species suitable as labels for labeled-entities include, but are not limited to, fluorescent dyes, e.g. Alexa Fluor® dyes such as Alexa Fluor® 647, Alexa Fluor® 488, quantum dots, optical dyes, luminescent dyes, and radionuclides, e.g. ¹²⁵I. Additional labels are described in further detail below.

The term “size exclusion chromatography” or “SEC” includes a chromatographic method in which molecules in solution are separated based on their size and/or hydrodynamic volume. It is applied to large molecules or macromolecular complexes such as proteins and their conjugates. Typically, when an aqueous solution is used to transport the sample through the column, the technique is known as gel filtration chromatography.

The terms “TNF inhibitor”, “TNF-α inhibitor,” “TNFα inhibitor,” “TNFα inhibitor agent” and “anti TNFα drug” are intended to encompass agents including peptides, proteins, antibodies, antibody fragments, fusion proteins (e.g., Ig fusion proteins or Fc fusion proteins), multivalent binding proteins (e.g., DVD Ig), small molecule TNF-α antagonists and similar naturally- or nonnaturally-occurring molecules, and/or recombinant and/or engineered forms thereof, that, directly or indirectly, inhibits TNF a activity, such as by inhibiting interaction of TNF-α with a cell surface receptor for TNF-α, inhibiting TNF-α protein production, inhibiting TNF-α gene expression, inhibiting TNFα secretion from cells, inhibiting TNF-α receptor signaling or any other means resulting in decreased TNF-α activity in a subject. The term “TNFα inhibitor” preferably includes agents which interfere with TNF-α activity. Examples of TNF-α inhibitors (anti-TNFα drug) include etanercept (ENBREL™, Amgen), infliximab (REMICADE™, Johnson and Johnson), human anti-TNF monoclonal antibody adalimumab (D2E7/HUMIRA™, Abbott Laboratories), certolizumab pegol (CIMZIA®, UCB, Inc.), golimumab (SIMPONI®; CNTO 148), CDP 571 (Celltech), and CDP 870 (Celltech), as well as other compounds which inhibit TNF-α activity, such that when administered to a subject suffering from or at risk of suffering from a disorder in which TNF-α activity is detrimental (e.g., IBD), the disorder is treated.

The terms “growth factor driven epithelial signaling inhibitor” and “growth factor driven epithelial signaling inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule growth factor driven epithelial signaling molecule antagonists and similar naturally- or nonnaturally-occurring molecules, and/or recombinant and/or engineered forms thereof, that, directly or indirectly, inhibits growth factor driven epithelial signaling activity, such as by inhibiting interaction of growth factor driven epithelial signaling receptor with its cognate growth factor or downstream signaling molecule, inhibiting growth factor driven epithelial signaling molecule protein production, inhibiting growth factor driven epithelial signaling molecule gene expression, inhibiting growth factor driven epithelial signaling molecule or any other means resulting in decreased growth factor driven epithelial signaling molecule activity in a subject. The term “growth factor driven epithelial signaling inhibitor” preferably includes agents which interfere with growth factor driven epithelial signaling (e.g., HER1, HER2, HER3, cMET, IGF-1R and the like) activity. Non-limiting examples of growth factor driven epithelial signaling inhibitors include pan-HER inhibitors such as PF-00299804, neratinib (HKI-272), AC480 (BMS-599626), BMS-690154, PF-02341066, HM781-36B, CI-1033, and BMW-2992; HER2 inhibitors including monoclonal antibodies such as trastuzumab (Herceptin®) and pertuzumab (2C4); small molecule tyrosine kinase inhibitors such as gefitinib (Iressa®), erlotinib (Tarceva®), pelitinib, CP-654577, CP-724714, canertinib (CI 1033), HKI-272, lapatinib (GW-572016; Tykerb®), PKI-166, AEE788, BMS-599626, HKI-357, BMW 2992, ARRY-334543, JNJ-26483327, and JNJ-26483327; and c-Met inhibitors including monoclonal antibodies such as AMG102 and MetMAb; small molecule inhibitors of c-Met such as ARQ197, JNJ-38877605, PF-04217903, SGX523, GSK 1363089/XL880, XL184, MGCD265, and MK-246, as well as other compounds which inhibit growth factor driven epithelial signaling activity, such that when administered to a subject suffering from or at risk of suffering from a disorder in which growth factor driven epithelial signaling activity is detrimental (e.g., IBD), the disorder is treated.

The terms “JAK inhibitor,” “JAK inhibitor agent” and “JAK inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule JAK antagonists and similar naturally- or nonnaturally-occurring molecules, and/or recombinant and/or engineered forms thereof, that, directly or indirectly, inhibits JAK and/or STAT activity (e.g., phosphorylation activity, such as by inhibiting interaction of a cell surface receptor (e.g., IL-2R family, IL-3R family, IL-6R family and IFN-R family receptors) with JAK, inhibiting JAK mediated activation (e.g., phosphorylation) of STAT protein, inhibiting JAK protein production, inhibiting JAK gene expression, inhibiting JAK secretion from cells, inhibiting JAK and/or STAT signaling or any other means resulting in decreased JAK activity and/or STAT activity in a subject. The term “JAK inhibitor” preferably includes agents which interfere with JAK activity. Non-limiting examples of JAK inhibitors (e.g., drugs) include tofacitinib (Xeljanz®, Pfizer), ruxolitinib (Jakafi®, Incyte Corp.), PRT2070 (a dual Syk-JAK inhibitor, Portola Pharmaceuticals), SAR302503 (Sanofi-Aventis), GLP0634 (Galapagos), and INCB39110 (Incyte Corp.), as well as other compounds which inhibit JAK activity, such that when administered to a subject suffering from or at risk of suffering from a disorder in which JAK activity is detrimental (e.g., IBD), the disorder is treated.

The terms “PI3K inhibitor,” “PI3K inhibitor agent” and “PI3K inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule PI3K antagonists and similar naturally- or nonnaturally-occurring molecules, and/or recombinant and/or engineered forms thereof, that, directly or indirectly, inhibits PI3K activity (e.g., phosphorylation activity, such as by inhibiting interaction of a cell surface receptor, inhibiting PI3K mediated activation (e.g., phosphorylation) of a substrate protein, inhibiting PI3K protein production, inhibiting PI3K gene expression, inhibiting PI3K secretion from cells, inhibiting PI3K signaling or any other means resulting in decreased PI3K activity in a subject. Non-limiting examples of PI3K inhibitors (e.g., drugs) include PX-866, wortmannin, LY 294002, quercetin, tetrodotoxin citrate, thioperamide maleate, GDC-0941 (957054-30-7), IC87114, PI-103, PIK93, BEZ235 (NVP-BEZ235), TGX-115, ZSTK474, (−)-deguelin, NU 7026, myricetin, tandutinib, GDC-0941 bismesylate, GSK690693, KU-55933, MK-2206, OSU-03012, perifosine, triciribine, XL-147, PIK75, TGX-221, NU 7441, PI 828, XL-765, and WHI-P 154, as well as other compounds which inhibit PI3K activity, such that when administered to a subject suffering from or at risk of suffering from a disorder in which PI3K activity is detrimental (e.g., IBD), the disorder is treated.

The terms “AKT inhibitor,” “AKT inhibitor agent” and “AKT inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule AKT antagonists and similar naturally- or nonnaturally-occurring molecules, and/or recombinant and/or engineered forms thereof, that, directly or indirectly, inhibits AKT activity (e.g., phosphorylation activity, such as by inhibiting interaction of a cell surface receptor, inhibiting AKT mediated activation (e.g., phosphorylation) of a substrate protein, inhibiting AKT protein production, inhibiting AKT gene expression, inhibiting AKT secretion from cells, inhibiting AKT signaling or any other means resulting in decreased AKT activity in a subject. Non-limiting examples of AKT inhibitors (e.g., drugs) include 1L6-hydroxymethyl-chiro-inositol-2-(R)-2-O-methyl-3-O-octadecyl-sn-glycerocarbonate, 9-methoxy-2-methylellipticinium acetate, 1,3-dihydro-1-(1-((4-(6-phenyl-1H-imidazo[4,5-g]quinoxalin-7-yl)phenyl)methyl)-4-piperidinyl)-2H-benzimidazol-2-one, 10-(4′-(N-diethylamino)butyl)-2-chlorophenoxazine, 3-formylchromone thiosemicarbazone (Cu(II)Cl₂ complex), API-2, a 15-mer peptide derived from amino acids 10-24 of the proto-oncogene TCL1 (Hiromura et al., J. Biol. Chem., 279:53407-53418 (2004), KP372-1, and the compounds described in Kozikowski et al., J. Am. Chem. Soc., 125:1144-1145 (2003) and Kau et al., Cancer Cell, 4:463-476 (2003), as well as other compounds which inhibit AKT activity, such that when administered to a subject suffering from or at risk of suffering from a disorder in which AKT activity is detrimental (e.g., IBD), the disorder is treated.

The terms “ERK inhibitor,” “ERK inhibitor agent” and “ERK inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule ERK antagonists and similar naturally- or nonnaturally-occurring molecules, and/or recombinant and/or engineered forms thereof, that, directly or indirectly, inhibits RAS-RAF-MEK-ERK pathway activity (e.g., phosphorylation activity, such as by inhibiting interaction of a cell surface receptor, inhibiting ERK mediated activation (e.g., phosphorylation) of a substrate protein, inhibiting ERK and/or MEK protein production, inhibiting ERK and/or MEK gene expression, inhibiting ERK signaling or any other means resulting in decreased ERK activity in a subject. Non-limiting examples of ERK inhibitors include Raf-1 inhibitors, such as sorafenib (Nexavar™, GlaxoSmithKline), dabrafenib (Tafinlar™, GlaxoSmithKline), XL281 (Exelixis), SB90885RAF265 (Novartis), GW5074, BAY 43-9006 (Bayer Healthcare Pharmaceuticals), and ISIS 5132 (ISIS Therapeutics); B-RAF inhibitor such as PLX4720 and vemurafenib (Zelboraf®, Genentech); MEK1/2 inhibitors, such as BAY86-9766 (Bayer Healthcare Pharmaceuticals), MEK162 (e.g., ARRY-162, Novartis), G-573 (Genentech), GDC-0623 (Genentech), GSK1120212 (GlaxoSmithKline), PD98059 (Pfizer), PD184352 (Pfizer), PD0325901 (Pfizer), U0126, AS703026/MSC1935369 (Merck), selumetinib (AZD6244/ARRY-142886, AstraZeneca) and trametinib (Mekini St™, GlaxoSmithKline); and MAPK pathway inhibitors such as SCH772984 (Merck), as well as other compounds which inhibit ERK pathway activity, such that when administered to a subject suffering from or at risk of suffering from a disorder in which ERK activity is detrimental (e.g., IBD), the disorder is treated.

The terms “MEK inhibitor,” “MEK inhibitor agent” and “MEK inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule MEK antagonists and similar naturally- or nonnaturally-occurring molecules, and/or recombinant and/or engineered forms thereof, that, directly or indirectly, inhibits MEK activity (e.g., phosphorylation activity, such as by inhibiting interaction of a cell surface receptor, inhibiting MEK mediated activation (e.g., phosphorylation) of a substrate protein, inhibiting MEK protein production, inhibiting MEK gene expression, inhibiting MEK secretion from cells, inhibiting MEK signaling or any other means resulting in decreased MEK activity in a subject. Non-limiting examples of MEK inhibitors include PD98059, ARRY-162, RDEA119, U0126, GDC-0973, PD184161, AZD6244, AZD8330, PD0325901, and ARRY-142886, as well as other compounds which inhibit MEK activity, such that when administered to a subject suffering from or at risk of suffering from a disorder in which MEK activity is detrimental (e.g., IBD), the disorder is treated.

The terms “mTOR inhibitor,” “mTOR inhibitor agent” and “mTOR inhibitor drug” are intended to encompass agents including peptides, proteins, antibodies, antibody fragments, fusion proteins (e.g., Ig fusion proteins or Fc fusion proteins), multivalent binding proteins (e.g., DVD Ig), small molecule mTOR antagonists and similar naturally- or nonnaturally-occurring molecules, and/or recombinant and/or engineered forms thereof, that, directly or indirectly, inhibits mTOR activity (e.g., phosphorylation activity, such as by inhibiting interaction of a cell surface receptor, inhibiting mTOR mediated activation (e.g., phosphorylation) of a substrate protein, inhibiting mTOR protein production, inhibiting mTOR gene expression, inhibiting mTOR secretion from cells, inhibiting mTOR signaling or any other means resulting in decreased mTOR activity in a subject.

The term “immunosuppressive drug” or “immunosuppressive agent” includes any substance capable of producing an immunosuppressive effect, e.g., the prevention or diminution of the immune response, as by irradiation or by administration of drugs such as anti-metabolites, anti-lymphocyte sera, antibodies, etc. Examples of immunosuppressive drugs include, without limitation, thiopurine drugs such as azathioprine (AZA) and metabolites thereof; anti-metabolites such as methotrexate (MTX); sirolimus (rapamycin); temsirolimus; everolimus; tacrolimus (FK-506); FK-778; anti-lymphocyte globulin antibodies, anti-thymocyte globulin antibodies, anti-CD3 antibodies, anti-CD4 antibodies, and antibody-toxin conjugates; cyclosporine; mycophenolate; mizoribine monophosphate; scoparone; glatiramer acetate; metabolites thereof; pharmaceutically acceptable salts thereof; derivatives thereof prodrugs thereof; and combinations thereof.

The term “thiopurine drug” includes azathioprine (AZA), 6-mercaptopurine (6-MP), or any metabolite thereof that has therapeutic efficacy and includes, without limitation, 6-thioguanine (6-TG), 6-methylmercaptopurine riboside, 6-thioinosine nucleotides (e.g., 6-thioinosine monophosphate, 6-thioinosine diphosphate, 6-thioinosine triphosphate), 6-thioguanine nucleotides (e.g., 6-thioguanosine monophosphate, 6-thioguanosine diphosphate, 6-thioguanosine triphosphate), 6-thioxanthosine nucleotides (e.g., 6-thioxanthosine monophosphate, 6-thioxanthosine diphosphate, 6-thioxanthosine triphosphate), derivatives thereof, analogues thereof, and combinations thereof.

The term “combination therapy” refers to a treatment regimen including more than one therapeutic agent, e.g., an anti-TNFα drug, a growth factor driven epithelial signaling inhibitor drug, a JAK inhibitor drug, a PI3K inhibitor drug, an AKT inhibitor drug, a MEK inhibitor drug, an mTOR inhibitor drug, another anti-signaling agent, an immunosuppressive drug, an anti-inflammatory drug, antibiotics, and a thiopurine drug. In some instances, the treatment regimen can also include surgery.

The term “predicting responsiveness to a therapeutic agent” is intended to refer to an ability to assess the likelihood that treatment of a subject with a therapeutic agent will or will not be effective in (e.g., provide a measurable benefit to) the subject. In particular, such an ability to assess the likelihood that treatment will or will not be effective typically is exercised after treatment has begun, and an indicator of effectiveness (e.g., an indicator of measurable benefit) has been observed in the subject. Particularly preferred anti-TNFα drugs, growth factor driven epithelial signaling inhibitor drugs, JAK inhibitor drugs, PI3K inhibitor drugs, AKT inhibitor drugs, ERK inhibitor drugs, MEK inhibitor drugs, mTOR inhibitor drugs are biologic agents that have been approved by the FDA for use in humans in the treatment of IBD (e.g., Crohn's disease and ulcerative colitis) and include those therapeutic agents described herein.

The term “course of therapy” includes any therapeutic approach taken to relieve or prevent one or more symptoms associated with IBD. The term encompasses administering any compound, drug, procedure, and/or regimen useful for improving the health of an individual with IBD and includes any of the therapeutic agents described herein. One skilled in the art will appreciate that either the course of therapy or the dose of the current course of therapy can be changed (e.g., increased or decreased) based upon the presence or concentration level of TNFα, anti-TNFα drug, anti-drug antibody and/or another therapeutic agent using the methods of the present invention.

### III. Detailed Description of the Embodiments

In some aspects, the present invention provides a method for determining whether a subject having inflammatory bowel disease (IBD) has non-inflamed and/or non-involved or inflamed and/or involved gastrointestinal tissue, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) comparing the total level and/or activation level of the one or more analytes measured in step (a) to that of a control, thereby determining whether the subject has non-inflamed and/or non-involved or inflamed and/or involved gastrointestinal tissue.

In some embodiments, step (a) comprises measuring the total level and/or activation level of any combination of two, three, four, five, six, seven, eight, nine, ten, eleven, twelve, thirteen, fourteen, or fifteen of the analytes. In other embodiments, the IBD is Crohn's disease (CD) or ulcerative colitis (UC).

In some embodiments, the total level of one or more analytes selected from the group consisting of HER1, HER2, cMET, and a combination thereof in the cell lysate is higher than that of the control, thereby determining that the subject has non-inflamed and/or non-involved gastrointestinal tissue. In other embodiments, the activation level of HER3 in the cell lysate is higher than that of the control, thereby determining that the subject has non-inflamed and/or non-involved gastrointestinal tissue. In particular embodiments, the control is an inflamed and/or involved gastrointestinal tissue, e.g., from a control subject having IBD (e.g., Crohn's disease).

In some embodiments, the total level of one or more analytes selected from the group consisting of HER1, HER2, cMET, and a combination thereof in the cell lysate is lower than that of the control, thereby determining that the subject has inflamed and/or involved gastrointestinal tissue and/or aggressive IBD. In other embodiments, the activation level of HER3 in the cell lysate is lower than that of the control, thereby determining that the subject has inflamed and/or involved gastrointestinal tissue and/or aggressive IBD. In yet other embodiments, a ratio of the total level of TNFα to the total level of HER2 (i.e., TNFα/HER2) in the cell lysate is higher than that of the control and/or a ratio of the total level of the anti-TNFα drug to the total level of HER2 (i.e., Drug/HER2) in the cell lysate is higher than that of the control, thereby determining that the subject has inflamed and/or involved gastrointestinal tissue and/or aggressive IBD. In particular embodiments, the control is a non-inflamed and/or non-involved gastrointestinal tissue, e.g., from a control subject having IBD (e.g., Crohn's disease).

In some instances, the subject is determined to have inflamed gastrointestinal tissue. In other instances, the subject is determined to have involved gastrointestinal tissue. In yet other instances, the subject is determined to have inflamed and involved gastrointestinal tissue. In certain instances, the subject is determined to have non-inflamed gastrointestinal tissue. In other instances, the subject is determined to have non-involved gastrointestinal tissue. In yet other instances, the subject is determined to have non-inflamed and non-involved gastrointestinal tissue. In further instances, the subject is determined to have non-inflamed and involved (e.g., previously involved) gastrointestinal tissue.

In certain embodiments, the gastrointestinal tissue sample is isolated from a subject using endoscopic ultrasound and fine needle aspiration (EUS/FNA). In other embodiments, the activation level of the analyte corresponds to the phosphorylation level thereof.

In some embodiments, the method further comprises calculating the total level and/or activation level of one or more analytes selected from the group consisting of HER1, HER2, HER3, cMET, IGF-1R, PI3K, AKT, PRAS40, MEK, RSK, JAK1, STAT1, STATS, TNFα, anti-TNFα drug, and combinations thereof. In some instances, the activated level of an analyte is divided by the total level of the same analyte, e.g., the level of activated HER3 is divided by the total level of HER3.

In certain embodiments, step (a) comprises measuring the total (e.g., expression) level of HER1, HER2, or cMET in the cell lysate. In other embodiments, step (a) comprises measuring the activation (e.g., phosphorylation) level of HER3 in the cell lysate. In some instances, step (a) comprises measuring the total level of HER1 and HER2, HER1 and cMET, HER2 and cMET, or HER1, HER2, and cMET in the cell lysate. In other instances, step (a) comprises measuring the total level of HER1, HER2, or cMET in the cell lysate in combination with measuring the activation level of HER3 in the cell lysate. In yet other instances, step (a) comprises measuring the total level of HER1 and HER2, HER1 and cMET, HER2 and cMET, or HER1, HER2, and cMET in the cell lysate in combination with measuring the activation level of HER3 in the cell lysate.

In certain embodiments, step (a) comprises measuring the total (e.g., expression) level of HER2 and TNFα, HER2 and anti-TNFα drug, or HER2, TNFα, and anti-TNFα drug in the cell lysate. In some instances, step (a) further comprises measuring the total level of HER1 and/or cMET in the cell lysate and/or measuring the activation level of HER3 in the cell lysate.

In some embodiments, step (a) comprises performing a proximity dual detection assay, an immunoassay, or a homogenous mobility shift assay (HMSA). In some instances, the proximity dual detection assay is a Collaborative Enzyme Enhanced Reactive Immunoassay (CEER™). In some instances, the immunoassay is an enzyme-linked immunosorbent assay (ELISA).

In some embodiments, the gastrointestinal tissue sample is isolated from a subject receiving IBD therapy. In certain instances, the gastrointestinal tissue sample is inflamed and/or involved tissue. In other instances, the IBD therapy is an anti-TNFα drug.

In some embodiments, the method for determining whether a subject having IBD has non-inflamed and/or non-involved or inflamed and/or involved gastrointestinal tissue further comprises selecting a suitable or optimal IBD therapy based upon the total level and/or activation level of one or more analytes measured in step (a).

As such, in related aspects, the present invention provides a method for selecting a suitable or optimal IBD therapy for the treatment of a subject having IBD, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) selecting a suitable or optimal IBD therapy for the subject having IBD based upon the total level and/or activation level calculated in step (a) compared to that of a control.

In some embodiments, the IBD therapy is selected from the group consisting of an anti-TNFα drug, a growth factor-driven epithelial signaling inhibitor drug, a JAK inhibitor drug, a PI3K inhibitor drug, an AKT inhibitor drug, an ERK inhibitor drug, a MEK inhibitor drug, an mTOR inhibitor drug, and a combination thereof. In other embodiments, the control is a gastrointestinal tissue sample taken from the subject prior to receiving an IBD therapy.

In certain instances, the suitable or optimal IBD therapy comprises an anti-TNFα drug if the total level and/or activation level of one or more of analytes selected from the group consisting of HER1, HER2, HER3, cMET, IGF-1R, and a combination thereof is lower than that of the control.

In certain instances, the suitable or optimal IBD therapy comprises a growth factor-driven epithelial signaling inhibitor drug if the total level and/or activation level of one or more of analytes selected from the group consisting HER1, HER2, HER3, cMET, IGF-1R, and a combination thereof is higher than that of the control. In other instances, the suitable or optimal IBD therapy comprises a JAK inhibitor drug if the activation level of STAT3 is higher than that of the control. In yet other instances, the suitable or optimal IBD therapy is selected from the group consisting of a PI3K inhibitor drug, an AKT inhibitor drug, an ERK inhibitor drug, a MEK inhibitor drug, an mTOR inhibitor drug, and a combination thereof if the activation level of one or more of analytes selected from the group consisting PI3K, AKT, PRAS40, MEK, RSK, and a combination thereof is higher than that of the control. In these instances, the gastrointestinal tissue sample can be taken from the colon of the subject and the control can comprise a non-inflamed and/or non-involved ileal tissue, e.g., from a control subject having IBD (e.g., Crohn's disease).

In some embodiments, the method further comprises applying a statistical analysis to the total level and/or activation level of the one or more analytes measured in step (a) to determine if the subject has non-inflamed and/or non-involved or inflamed and/or involved gastrointestinal tissue or to select a suitable or optimal IBD therapy. In certain embodiments, the statistical analysis comprises a quartile analysis to obtain a quartile sum score. In other embodiments, the statistical analysis comprises a multiple logistic regression model.

In particular embodiments, the method for determining whether a subject having IBD has non-inflamed and/or non-involved or inflamed and/or involved gastrointestinal tissue or the method for selecting a suitable or optimal IBD therapy for the treatment of a subject having IBD is performed at a plurality of time points to monitor the progression of mucosal healing, i.e., monitoring the transition through the phases of mucosal healing from the inflammatory phase, proliferation phase, and remodeling phase towards complete repair of the intestinal mucosa. In particular embodiments, a transition from an inflamed and/or involved gastrointestinal tissue determined at an earlier time point (e.g., at diagnosis or at initiation of a course of therapy for IBD) to a non-inflamed and/or non-involved gastrointestinal tissue determined at a later time point (e.g., at any point during a course of therapy for IBD) indicates that the subject is undergoing or has undergone mucosal healing.

In other aspects, the present invention provides a method for monitoring the therapeutic response to an IBD therapy in a subject having IBD, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) determining whether the subject is responding to the IBD therapy based upon the total expression level and/or activation level of one or more analytes measured in step (a) compared to that of a control.

In some embodiments, step (a) comprises measuring the total level and/or activation level of any combination of two, three, four, five, six, seven, eight, nine, ten, eleven, twelve, thirteen, fourteen, or fifteen of the analytes. In other embodiments, the IBD is Crohn's disease (CD) or ulcerative colitis (UC).

In some embodiments, the IBD therapy is selected from the group consisting of an anti-TNFα drug, a growth factor-driven epithelial signaling inhibitor drug, a JAK inhibitor drug, a PI3K inhibitor drug, an AKT inhibitor drug, an ERK inhibitor drug, a MEK inhibitor drug, an mTOR inhibitor drug, and a combination thereof. In other embodiments, the control is a gastrointestinal tissue sample taken from the subject prior to receiving an IBD therapy.

In certain embodiments, the subject is determined to be responding to the IBD therapy if the total level of HER1, HER2, or cMET and/or the activation level of HER3 is equal to or higher compared to that of the control, wherein the control is gastrointestinal tissue from a healthy subject. In other embodiments, the subject is determined to be responding to the IBD therapy if a ratio of the total level of TNFα to the total level of HER2 (i.e., TNFα/HER2) is equal to or lower than that of the control and/or a ratio of the total level of the anti-TNFα drug to the total level of HER2 (i.e., Drug/HER2) is equal to or lower than that of the control, wherein the control is gastrointestinal tissue from a healthy subject.

In certain embodiments, the subject is determined to be responding to the IBD therapy if the total level of HER1, HER2, or cMET and/or the activation level of HER3 is higher compared to that of the control, wherein the control is inflamed and/or involved gastrointestinal tissue. In other embodiments, the subject is determined to be responding to the IBD therapy if a ratio of the total level of TNFα to the total level of HER2 (i.e., TNFα/HER2) is lower than that of the control and/or a ratio of the total level of the anti-TNFα drug to the total level of HER2 (i.e., Drug/HER2) is lower than that of the control, wherein the control is inflamed and/or involved gastrointestinal tissue.

In certain embodiments, step (a) comprises measuring the total (e.g., expression) level of HER1, HER2, or cMET in the cell lysate. In other embodiments, step (a) comprises measuring the activation (e.g., phosphorylation) level of HER3 in the cell lysate. In some instances, step (a) comprises measuring the total level of HER1 and HER2, HER1 and cMET, HER2 and cMET, or HER1, HER2, and cMET in the cell lysate. In other instances, step (a) comprises measuring the total level of HER1, HER2, or cMET in the cell lysate in combination with measuring the activation level of HER3 in the cell lysate. In yet other instances, step (a) comprises measuring the total level of HER1 and HER2, HER1 and cMET, HER2 and cMET, or HER1, HER2, and cMET in the cell lysate in combination with measuring the activation level of HER3 in the cell lysate.

In certain embodiments, step (a) comprises measuring the total (e.g., expression) level of HER2 and TNFα, HER2 and anti-TNFα drug, or HER2, TNFα, and anti-TNFα drug in the cell lysate. In some instances, step (a) further comprises measuring the total level of HER1 and/or cMET in the cell lysate and/or measuring the activation level of HER3 in the cell lysate.

In certain embodiments, the gastrointestinal tissue sample is isolated from a subject using endoscopic ultrasound and fine needle aspiration (EUS/FNA). In other embodiments, the activation level of the analyte corresponds to the phosphorylation level thereof.

In some embodiments, step (a) comprises performing a proximity dual detection assay, an immunoassay, or a homogenous mobility shift assay (HMSA). In some instances, the proximity dual detection assay is a Collaborative Enzyme Enhanced Reactive Immunoassay (CEER™). In some instances, the immunoassay is an enzyme-linked immunosorbent assay (ELISA).

In some embodiments, the method further comprises applying a statistical analysis to the total level and/or activation level of the one or more analytes measured in step (a) to monitor the therapeutic response to an IBD therapy. In certain embodiments, the statistical analysis comprises a quartile analysis to obtain a quartile sum score. In other embodiments, the statistical analysis comprises a multiple logistic regression model.

In particular embodiments, the method for monitoring the therapeutic response to an IBD therapy in a subject having IBD is performed at a plurality of time points to monitor the progression of mucosal healing, i.e., monitoring the transition through the phases of mucosal healing from the inflammatory phase, proliferation phase, and remodeling phase towards complete repair of the intestinal mucosa. In particular embodiments, a transition from an inflamed and/or involved gastrointestinal tissue determined at an earlier time point (e.g., at diagnosis or at initiation of a course of therapy for IBD) to a non-inflamed and/or non-involved gastrointestinal tissue determined at a later time point (e.g., at any point during a course of therapy for IBD) indicates that the subject is undergoing or has undergone mucosal healing.

In further aspects, the present invention provides a method for diagnosing early onset inflammatory bowel disease (IBD) in a subject, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) comparing the total level and/or activation level of the one or more analytes measured in step (a) to that of a control, thereby determining that the subject has early onset IBD if the total level and/or activation level of the one or more analytes is higher compared to that of the control.

In some embodiments, step (a) comprises measuring the total level and/or activation level of any combination of two, three, four, five, six, seven, eight, nine, ten, eleven, twelve, thirteen, fourteen, or fifteen of the analytes. In other embodiments, the IBD is Crohn's disease (CD) or ulcerative colitis (UC).

In certain embodiments, the control is an inflamed and/or involved gastrointestinal tissue sample from a subject having IBD. In some instances, the subject has early onset IBD if the total level of TNFα is higher and/or the total level or activation level of one or more analytes selected from the group consisting of HER1, HER2, HER3, cMET, IGF-1R, PI3K, AKT, PRAS40, MEK, RSK, JAK1, STAT1, STAT3, and a combination thereof is lower than the control.

In some embodiments, the gastrointestinal tissue sample is isolated from a subject using endoscopic ultrasound and fine needle aspiration (EUS/FNA). In other embodiments, the activation level of the analyte corresponds to the phosphorylation level thereof.

In some embodiments, step (a) comprises performing a proximity dual detection assay, an immunoassay, or a homogenous mobility shift assay (HMSA). In some instances, the proximity dual detection assay is a Collaborative Enzyme Enhanced Reactive Immunoassay (CEER™). In some instances, the immunoassay is an enzyme-linked immunosorbent assay (ELISA).

In some embodiments, the method further comprises applying a statistical analysis to the total level and/or activation level of the one or more analytes measured in step (a) to diagnose early onset IBD. In certain embodiments, the statistical analysis comprises a quartile analysis to obtain a quartile sum score. In other embodiments, the statistical analysis comprises a multiple logistic regression model.

In other aspects, the present invention provides a method for diagnosing ulcerative colitis (UC) in a subject, the method comprising:
(a) measuring the total level and/or activation level of STAT3 in a colon cell lysate, wherein the colon cell lysate is produced by lysing a cell from a colon sample taken from the subject;
(b) measuring the total level and/or activation level of STAT3 in an ileum cell lysate, wherein the ileum cell lysate is produced by lysing a cell from an ileum sample taken from the subject; and
(c) comparing the total level and/or activation level of STAT3 in the colon cell lysate to that of the ileum cell lysate, thereby diagnosing UC in the subject.

In some embodiments, the colon sample and the ileum sample are inflamed and/or involved tissues. In other embodiments, the colon sample and the ileum sample are non-inflamed and/or non-involved tissues.

In some embodiments, the colon sample is isolated from a subject using endoscopic ultrasound and fine needle aspiration (EUS/FNA). In some embodiments, the ileum sample is isolated from a subject using endoscopic ultrasound and fine needle aspiration (EUS/FNA). In other embodiments, the activation level of STAT3 corresponds to the phosphorylation level thereof.

In some embodiments, step (a) and/or step (b) comprises performing a proximity dual detection assay or an immunoassay. In some instances, the proximity dual detection assay is a Collaborative Enzyme Enhanced Reactive Immunoassay (CEER™). In some instances, the immunoassay is an enzyme-linked immunosorbent assay (ELISA).

In some embodiments, the diagnosis is indicative of UC when STAT3 activation is higher in the colon sample compared to the ileum sample. In other embodiments, the diagnosis is indicative of not having UC when STAT3 activation is equal to or lower in the colon sample compared to the ileum sample.

In certain embodiments, the method further comprises selecting a suitable UC therapy selected from the group consisting of an anti-TNFα drug, a JAK inhibitor drug, and a combination thereof.

A. Sample Isolation

In some embodiments, the gastrointestinal tissue (e.g., a gastrointestinal cell of the colon and/or a gastrointestinal cell of the ileum) of the method described herein is harvested from an individual by endoscopic ultrasound and fine needle aspiration (EUS/FNA). One skilled in the art recognizes that an EUS/FNA is a surgical procedure that removes gastrointestinal tissue. Typically, endoscopic ultrasound is used to locate a specific region of the individual's gastrointestinal tract and tools such as fine gauge needles, biopsy forceps, snares, detachable loop ligating devices are used to safely remove the gastrointestinal issue and collect it for analysis. Prior to EUS/FNA, the colon can be cleared and completely cleaned.

To preserve the in situ activation states, the analytes are typically extracted shortly after the tissue samples are isolated. Preferably the tissue samples are lysed within 96, 72, 48, 24, 6, or 1 hr, more preferably within 30, 15, or 5 minutes. The isolated cells may also be incubated with growth factors usually at nanomolar to micromolar concentrations for about 1-30 minutes to resuscitate or stimulate signal transducer activation (see, e.g., Irish et al., Cell, 118:217-228 (2004)). Stimulatory growth factors include epidermal growth factor (EGF), heregulin (HRG), TGF-α, PIGF, angiopoietin (Ang), NRG1, PGF, TNF-α, VEGF, PDGF, IGF, FGF, HGF, cytokines, and the like. After isolation (e.g., tissue biopsy by EUS/FNA), the cells are lysed to extract the analytes using any technique known in the art. Preferably, the cell lysis is initiated between about 1-360 minutes. In some embodiments, a commercially available lysis buffer such as, but not limited to, MSD lysis buffer (Meso Scale Discovery, Gaithersburg, Md.) is used to lyse cells in the tissue sample. In other embodiments, a protein preservation solution or buffer is used to lyse the cells. In some instances, the cells are lysed according to the manufacturer's instruction protocol. In some embodiments, the lysate is stored at −80° C. until use.

B. Analytes

In some embodiments, the methods of the present invention comprise determining the total expression level (e.g., total amount) and/or activation level (e.g., level of phosphorylation or complex formation) of at least one or more (e.g., any combination of two, three, four, five, six, seven, eight, nine, ten, eleven, twelve, thirteen, fourteen, or fifteen) of the following analytes in a cell lysate: (1) HER1/EGFR/ErbB1; (2) HER2/ErbB2 (including p95HER2 and/or other forms of truncated HER2); (3) HER3/ErbB3; (4) c-MET; (5) IGF-1R; (6) PI3K (e.g., PIK3CA and/or PIK3R1); (7) AKT; (8) PRAS40; (9) MEK; (10) RSK; (11) JAK1; (12) STAT1; (13) STAT3; (14) TNFα; (15) anti-TNFα drug; and a combination thereof.

In some embodiments, the activation level corresponds to a level of phosphorylation of HER1/EGFR/ErbB1, HER2/ErbB2 (including p95HER2 and/or other forms of truncated HER2), HER3/ErbB3, c-MET, IGF-1R, PI3K (e.g., PIK3CA and/or PIK3R1), AKT, PRAS40, MEK, RSK, JAK1, STAT1, STAT3 and a combination thereof. In other embodiments, the activation level corresponds to a level of a PI3K complex. Examples of PI3K complexes include, without limitation, one or more complexes comprising a dimerized receptor tyrosine kinase pair, a PI3K p85 subunit (e.g., PIK3R1), and a PI3K p110 subunit (e.g., an α or β subunit such as PIK3CA or PIK3CB); see, for example, International Patent Publication No. WO 2013/033623, the disclosure of which is herein incorporated by reference in its entirety for all purposes.

In certain embodiments, the present invention further comprises determining the total expression level (e.g., total amount) and/or activation level (e.g., level of phosphorylation or complex formation) of one or more (e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more) additional analytes in a cell lysate. In some embodiments, the one or more (e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more) additional analytes comprises one or more signal transduction molecules selected from the group consisting of receptor tyrosine kinases, non-receptor tyrosine kinases, signaling molecules, nuclear hormone receptors, nuclear receptor coactivators, nuclear receptor repressors, and combinations thereof. In yet other embodiments, the one or more additional analytes comprise a therapeutic agent such as a pharmaceutically effective drug.

C. Methods for Measuring Total Expression or Activation Levels of Analytes

In some embodiments, the assay for detecting the total expression and/or activation level of one or more analytes of interest in a cell lysate is a multiplex, high-throughput proximity (i.e., three-antibody) assay having superior dynamic range. As a non-limiting example, the three antibodies used in the proximity assay can comprise: (1) a capture antibody specific for a particular analyte of interest; (2) a detection antibody specific for an activated form of the analyte (i.e., activation state-dependent antibody); and (3) a detection antibody which detects the total amount of the analyte (i.e., activation state-independent antibody). The activation state-dependent antibody is capable of detecting, e.g., the phosphorylation, ubiquitination, and/or complexation state of the analyte, while the activation state-independent antibody is capable of detecting the total amount (i.e., both the activated and non-activated forms) of the analyte.

In particular embodiments, the proximity assay for detecting the activation level or status of an analyte of interest comprises:
(i) incubating the cell lysate with one or a plurality of dilution series of capture antibodies to form a plurality of captured analytes;
(ii) incubating the plurality of captured analytes with detection antibodies comprising one or a plurality of activation state-independent antibodies and one or a plurality of activation state-dependent antibodies specific for the corresponding analytes to form a plurality of detectable captured analytes,
wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair;
(iii) incubating the plurality of detectable captured analytes with a second member of the signal amplification pair to generate an amplified signal; and
(iv) detecting the amplified signal generated from the first and second members of the signal amplification pair.

In alternative embodiments, the activation state-dependent antibodies can be labeled with a facilitating moiety and the activation state-independent antibodies can be labeled with a first member of a signal amplification pair.

As another non-limiting example, the three antibodies used in the proximity assay can comprise: (1) a capture antibody specific for a particular analyte of interest; (2) a first detection antibody which detects the total amount of the analyte (i.e., a first activation state-independent antibody); and (3) a second detection antibody which detects the total amount of the analyte (i.e., a second activation state-independent antibody). In preferred embodiments, the first and second activation state-independent antibodies recognize different (e.g., distinct) epitopes on the analyte.

In particular embodiments, the proximity assay for detecting the total level of an analyte of interest comprises:
(i) incubating the cell lysate with one or a plurality of dilution series of capture antibodies to form a plurality of captured analytes;
(ii) incubating the plurality of captured analytes with detection antibodies comprising one or a plurality of first and second activation state-independent antibodies specific for the corresponding analytes to form a plurality of detectable captured analytes,
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(iii) incubating the plurality of detectable captured analytes with a second member of the signal amplification pair to generate an amplified signal; and
(iv) detecting the amplified signal generated from the first and second members of the signal amplification pair.

In alternative embodiments, the first activation state-independent antibodies can be labeled with a first member of a signal amplification pair and the second activation state-independent antibodies can be labeled with a facilitating moiety.

The proximity assays described herein are typically antibody-based arrays which comprise one or a plurality of different capture antibodies at a range of capture antibody concentrations that are coupled to the surface of a solid support in different addressable locations. Examples of suitable solid supports for use in the present invention are described above.

The capture antibodies, activation state-independent antibodies, and activation state-dependent antibodies are preferably selected to minimize competition between them with respect to analyte binding (i.e., all antibodies can simultaneously bind their corresponding signal transduction molecules).

In some embodiments, activation state-independent antibodies for detecting activation levels of one or more of the analytes or, alternatively, first activation state-independent antibodies for detecting total expression levels of one or more of the analytes further comprise a detectable moiety. In such instances, the amount of the detectable moiety is correlative to the amount of one or more of the analytes in the cell lysate. Examples of detectable moieties include, but are not limited to, fluorescent labels, chemically reactive labels, enzyme labels, radioactive labels, and the like. Preferably, the detectable moiety is a fluorophore such as an Alexa Fluor® dye (e.g., Alexa Fluor® 647), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The detectable moiety can be coupled directly or indirectly to the activation state-independent antibodies using methods well-known in the art.

In certain instances, activation state-independent antibodies for detecting activation levels of one or more of the analytes or, alternatively, first activation state-independent antibodies for detecting total expression levels of one or more of the analytes are directly labeled with the facilitating moiety. The facilitating moiety can be coupled to activation state-independent antibodies using methods well-known in the art. A suitable facilitating moiety for use in the present invention includes any molecule capable of generating an oxidizing agent which channels to (i.e., is directed to) and reacts with (i.e., binds, is bound by, or forms a complex with) another molecule in proximity (i.e., spatially near or close) to the facilitating moiety. Examples of facilitating moieties include, without limitation, enzymes such as glucose oxidase or any other enzyme that catalyzes an oxidation/reduction reaction involving molecular oxygen (O₂) as the electron acceptor, and photosensitizers such as methylene blue, rose bengal, porphyrins, squarate dyes, phthalocyanines, and the like. Non-limiting examples of oxidizing agents include hydrogen peroxide (H₂O₂), a singlet oxygen, and any other compound that transfers oxygen atoms or gains electrons in an oxidation/reduction reaction. Preferably, in the presence of a suitable substrate (e.g., glucose, light, etc.), the facilitating moiety (e.g., glucose oxidase, photosensitizer, etc.) generates an oxidizing agent (e.g., hydrogen peroxide (H₂O₂), single oxygen, etc.) which channels to and reacts with the first member of the signal amplification pair (e.g., horseradish peroxidase (HRP), hapten protected by a protecting group, an enzyme inactivated by thioether linkage to an enzyme inhibitor, etc.) when the two moieties are in proximity to each other.

In certain other instances, activation state-independent antibodies for detecting activation levels of one or more of the analytes or, alternatively, first activation state-independent antibodies for detecting total expression levels of one or more of the analytes are indirectly labeled with the facilitating moiety via hybridization between an oligonucleotide linker conjugated to the activation state-independent antibodies and a complementary oligonucleotide linker conjugated to the facilitating moiety. The oligonucleotide linkers can be coupled to the facilitating moiety or to the activation state-independent antibodies using methods well-known in the art. In some embodiments, the oligonucleotide linker conjugated to the facilitating moiety has 100% complementarity to the oligonucleotide linker conjugated to the activation state-independent antibodies. In other embodiments, the oligonucleotide linker pair comprises at least one, two, three, four, five, six, or more mismatch regions, e.g., upon hybridization under stringent hybridization conditions. One skilled in the art will appreciate that activation state-independent antibodies specific for different analytes can either be conjugated to the same oligonucleotide linker or to different oligonucleotide linkers.

The length of the oligonucleotide linkers that are conjugated to the facilitating moiety or to the activation state-independent antibodies can vary. In general, the linker sequence can be at least about 5, 10, 15, 20, 25, 30, 35, 40, 45, 50, 75, or 100 nucleotides in length. Typically, random nucleic acid sequences are generated for coupling. As a non-limiting example, a library of oligonucleotide linkers can be designed to have three distinct contiguous domains: a spacer domain; signature domain; and conjugation domain. Preferably, the oligonucleotide linkers are designed for efficient coupling without destroying the function of the facilitating moiety or activation state-independent antibodies to which they are conjugated.

The oligonucleotide linker sequences can be designed to prevent or minimize any secondary structure formation under a variety of assay conditions. Melting temperatures are typically carefully monitored for each segment within the linker to allow their participation in the overall assay procedures. Generally, the range of melting temperatures of the segment of the linker sequence is between 1-10° C. Computer algorithms (e.g., OLIGO 6.0) for determining the melting temperature, secondary structure, and hairpin structure under defined ionic concentrations can be used to analyze each of the three different domains within each linker. The overall combined sequences can also be analyzed for their structural characterization and their comparability to other conjugated oligonucleotide linker sequences, e.g., whether they will hybridize under stringent hybridization conditions to a complementary oligonucleotide linker.

The spacer region of the oligonucleotide linker provides adequate separation of the conjugation domain from the oligonucleotide crosslinking site. The conjugation domain functions to link molecules labeled with a complementary oligonucleotide linker sequence to the conjugation domain via nucleic acid hybridization. The nucleic acid-mediated hybridization can be performed either before or after antibody-analyte (i.e., antigen) complex formation, providing a more flexible assay format. Unlike many direct antibody conjugation methods, linking relatively small oligonucleotides to antibodies or other molecules has minimal impact on the specific affinity of antibodies towards their target analyte or on the function of the conjugated molecules.

In some embodiments, the signature sequence domain of the oligonucleotide linker can be used in complex multiplexed protein assays. Multiple antibodies can be conjugated with oligonucleotide linkers with different signature sequences. In multiplex immunoassays, reporter oligonucleotide sequences labeled with appropriate probes can be used to detect cross-reactivity between antibodies and their antigens in the multiplex assay format.

Oligonucleotide linkers can be conjugated to antibodies or other molecules using several different methods. For example, oligonucleotide linkers can be synthesized with a thiol group on either the 5′ or 3′ end. The thiol group can be deprotected using reducing agents (e.g., TCEP-HCl) and the resulting linkers can be purified by using a desalting spin column. The resulting deprotected oligonucleotide linkers can be conjugated to the primary amines of antibodies or other types of proteins using heterobifunctional cross linkers such as SMCC. Alternatively, 5′-phosphate groups on oligonucleotides can be treated with water-soluble carbodiimide EDC to form phosphate esters and subsequently coupled to amine-containing molecules. In certain instances, the diol on the 3′-ribose residue can be oxidized to aldehyde groups and then conjugated to the amine groups of antibodies or other types of proteins using reductive amination. In certain other instances, the oligonucleotide linker can be synthesized with a biotin modification on either the 3′ or 5′ end and conjugated to streptavidin-labeled molecules.

Oligonucleotide linkers can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). In general, the synthesis of oligonucleotides makes use of common nucleic acid protecting and coupling groups, such as dimethoxytrityl at the 5′-end and phosphoramidites at the 3′-end. Suitable reagents for oligonucleotide synthesis, methods for nucleic acid deprotection, and methods for nucleic acid purification are known to those of skill in the art.

In certain instances, activation state-dependent antibodies for detecting activation levels of one or more of the analytes or, alternatively, second activation state-independent antibodies for detecting total expression levels of one or more of the analytes are directly labeled with the first member of the signal amplification pair. The signal amplification pair member can be coupled to activation state-dependent antibodies to detect activation levels or second activation state-independent antibodies to detect expression levels using methods well-known in the art. In certain other instances, activation state-dependent antibodies or second activation state-independent antibodies are indirectly labeled with the first member of the signal amplification pair via binding between a first member of a binding pair conjugated to the activation state-dependent antibodies or second activation state-independent antibodies and a second member of the binding pair conjugated to the first member of the signal amplification pair. The binding pair members (e.g., biotin/streptavidin) can be coupled to the signal amplification pair member or to the activation state-dependent antibodies or second activation state-independent antibodies using methods well-known in the art. Examples of signal amplification pair members include, but are not limited to, peroxidases such horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, and the like. Other examples of signal amplification pair members include haptens protected by a protecting group and enzymes inactivated by thioether linkage to an enzyme inhibitor.

In one example of proximity channeling, the facilitating moiety is glucose oxidase (GO) and the first member of the signal amplification pair is horseradish peroxidase (HRP). When the GO is contacted with a substrate such as glucose, it generates an oxidizing agent (i.e., hydrogen peroxide (H₂O₂)). If the HRP is within channeling proximity to the GO, the H₂O₂ generated by the GO is channeled to and complexes with the HRP to form an HRP-H₂O₂ complex, which, in the presence of the second member of the signal amplification pair (e.g., a chemiluminescent substrate such as luminol or isoluminol or a fluorogenic substrate such as tyramide (e.g., biotin-tyramide), homovanillic acid, or 4-hydroxyphenyl acetic acid), generates an amplified signal. Methods of using GO and HRP in a proximity assay are described in, e.g., Langry et al., U.S. Dept. of Energy Report No. UCRL-ID-136797 (1999). When biotin-tyramide is used as the second member of the signal amplification pair, the HRP-H₂O₂ complex oxidizes the tyramide to generate a reactive tyramide radical that covalently binds nearby nucleophilic residues. The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor® dye (e.g., Alexa Fluor® 555), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS), 4-chloro-1-napthol (4CN), and/or porphyrinogen.

In another example of proximity channeling, the facilitating moiety is a photosensitizer and the first member of the signal amplification pair is a large molecule labeled with multiple haptens that are protected with protecting groups that prevent binding of the haptens to a specific binding partner (e.g., ligand, antibody, etc.). For example, the signal amplification pair member can be a dextran molecule labeled with protected biotin, coumarin, and/or fluorescein molecules. Suitable protecting groups include, but are not limited to, phenoxy-, analino-, olefin-, thioether-, and selenoether-protecting groups. Additional photosensitizers and protected hapten molecules suitable for use in the proximity assays of the present invention are described in U.S. Pat. No. 5,807,675. When the photosensitizer is excited with light, it generates an oxidizing agent (i.e., singlet oxygen). If the hapten molecules are within channeling proximity to the photosensitizer, the singlet oxygen generated by the photosensitizer is channeled to and reacts with thioethers on the protecting groups of the haptens to yield carbonyl groups (ketones or aldehydes) and sulphinic acid, releasing the protecting groups from the haptens. The unprotected haptens are then available to specifically bind to the second member of the signal amplification pair (e.g., a specific binding partner that can generate a detectable signal). For example, when the hapten is biotin, the specific binding partner can be an enzyme-labeled streptavidin. Exemplary enzymes include alkaline phosphatase, β-galactosidase, HRP, etc. After washing to remove unbound reagents, the detectable signal can be generated by adding a detectable (e.g., fluorescent, chemiluminescent, chromogenic, etc.) substrate of the enzyme and detected using suitable methods and instrumentation known in the art. Alternatively, the detectable signal can be amplified using tyramide signal amplification and the activated tyramide either directly detected or detected upon the addition of a signal-detecting reagent as described above.

In yet another example of proximity channeling, the facilitating moiety is a photosensitizer and the first member of the signal amplification pair is an enzyme-inhibitor complex. The enzyme and inhibitor (e.g., phosphonic acid-labeled dextran) are linked together by a cleavable linker (e.g., thioether). When the photosensitizer is excited with light, it generates an oxidizing agent (i.e., singlet oxygen). If the enzyme-inhibitor complex is within channeling proximity to the photosensitizer, the singlet oxygen generated by the photosensitizer is channeled to and reacts with the cleavable linker, releasing the inhibitor from the enzyme, thereby activating the enzyme. An enzyme substrate is added to generate a detectable signal, or alternatively, an amplification reagent is added to generate an amplified signal.

In a further example of proximity channeling, the facilitating moiety is HRP, the first member of the signal amplification pair is a protected hapten or an enzyme-inhibitor complex as described above, and the protecting groups comprise p-alkoxy phenol. The addition of phenylenediamine and H₂O₂ generates a reactive phenylene diimine which channels to the protected hapten or the enzyme-inhibitor complex and reacts with p-alkoxy phenol protecting groups to yield exposed haptens or a reactive enzyme. The amplified signal is generated and detected as described above (see, e.g., U.S. Pat. Nos. 5,532,138 and 5,445,944).

An exemplary protocol for performing the proximity assays described herein is provided in U.S. Pat. No. 8,163,499, the disclosure of which is herein incorporated by reference in its entirety for all purposes.

In another embodiment, the present invention provides kits for performing the proximity assays described above comprising: (a) a dilution series of one or a plurality of capture antibodies restrained on a solid support; and (b) one or a plurality of detection antibodies (e.g., a combination of activation state-independent antibodies and activation state-dependent antibodies for detecting activation levels and/or a combination of first and second activation state-independent antibodies for detecting expression levels). In some instances, the kits can further contain instructions for methods of using the kit to detect the expression and/or activation level of one or a plurality of signal transduction molecules of cells such as gastrointestinal cells. The kits may also contain any of the additional reagents described above with respect to performing the specific methods of the present invention such as, for example, first and second members of the signal amplification pair, tyramide signal amplification reagents, substrates for the facilitating moiety, wash buffers, etc.

In certain instances, CEER™ is performed on the sample and the amount of phosphorylated or activated signaling molecule can be assessed or measured. After measuring the activated amount, the measured value is compared to a defined range. If the measured amount appears in the upper part of the range, then the activated signaling molecule is a target for therapy.

In certain embodiments, a reference (control) expression or activation level of the one or more analytes is obtained from a normal cell such as a gastrointestinal cell from a healthy individual not having IBD. In other embodiments, a reference expression or activation level of the one or more analytes is obtained from a gastrointestinal cell from a patient with IBD having inflamed and/or involved gastrointestinal tissue.

One skilled in the art will appreciate that the control(s) for the present invention described herein are selected according to each method and/or each embodiment of the method. For example, if the method is to determine whether the subject having IBD also has inflamed and/or involved gastrointestinal tissue, one skilled in the art will recognize that the control (e.g., negative control) can be a normal cell from healthy individual not having IBD or the control (e.g., positive control) can be inflamed and/or involved tissue from a patient with IBD.

In some embodiments, the reference expression or activation level of the one or more analytes is obtained from a cell (e.g., a gastrointestinal cell such as a colon or ileum cell obtained from a sample) that is not treated with a therapeutic IBD drug. In particular embodiments, the cell that is not treated with the therapeutic IBD drug is obtained from the same sample that the isolated cell (e.g., a test cell to be interrogated) that is used to produce the cell lysate is obtained. In certain instances, the presence of a lower level of expression or activation of the one or more analytes compared to the reference expression or activation level indicates that the therapeutic agent is suitable for the treatment of IBD (e.g., the gastrointestinal cells of an IBD patient have an increased likelihood of response to the therapeutic IBD drug). In certain instances, the presence of an identical, similar, or higher level of total expression or activation of the one or more analytes compared to the reference (control) expression or activation level indicates that the therapeutic IBD drug is unsuitable for the treatment of IBD (e.g., the gastrointestinal cells of an IBD patient have an increased likelihood of response to the therapeutic IBD drug).

In alternative embodiments, the reference (control) expression or activation level of the one or more analytes is obtained from a cell sensitive to the therapeutic IBD drug that is treated with the therapeutic IBD drug. In such embodiments, the presence of an identical, similar, or lower level of total expression or activation of the one or more analytes compared to the reference expression or activation level indicates that the therapeutic IBD drug is suitable for the treatment of the IBD (e.g., the gastrointestinal cells of an IBD patient have an increased likelihood of response to the therapeutic IBD drug). In certain other alternative embodiments, the reference expression or activation level of the one or more analytes is obtained from a cell resistant to the therapeutic IBD drug that is treated with the therapeutic IBD drug. In such embodiments, the presence of an identical, similar, or higher level of total expression or activation of the one or more analytes compared to the reference (control) expression or activation level indicates that the therapeutic IBD drug is unsuitable for the treatment of the IBD (e.g., the gastrointestinal cells of an IBD patient have an increased likelihood of response to the therapeutic IBD drug).

In certain embodiments, a higher level of expression or activation of the one or more analytes is considered to be present in a cell or cell lysate when the expression or activation level is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or 100-fold higher (e.g., about 1.5-3, 2-3, 2-4, 2-5, 2-10, 2-20, 2-50, 3-5, 3-10, 3-20, 3-50, 4-5, 4-10, 4-20, 4-50, 5-10, 5-15, 5-20, or 5-50-fold higher) than the reference expression or activation level of the corresponding analyte in a cell from non-inflamed and/or non-involved gastrointestinal tissue, in a cell from inflamed and/or involved gastrointestinal tissue, in a cell not treated with a therapeutic IBD drug, in a therapeutic IBD drug-sensitive cell treated with the therapeutic IBD drug, or in a therapeutic IBD drug-resistant cell treated with the therapeutic IBD drug. In some embodiments, a higher level of total expression or activation of the one or more analytes (e.g., epithelial cell analytes such as HER1, HER2, HER3, cMET, IGF-1R, PI3K, AKT, PRAS40, MEK, RSK, JAK1, STAT1, and STAT3) compared to the reference (e.g., a sample from a control patient with IBD) indicates that the individual has non-inflamed or non-involved gastrointestinal tissue. In some instances, the individual may be undergoing or have undergone mucosal healing. In certain embodiments, an equal or higher total expression level of TNFα relative to the control indicates that the individual has inflamed and/or involved tissue.

In other embodiments, a lower level of expression or activation of the one or more analytes is considered to be present in a cell or cell lysate when the expression or activation level is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or 100-fold lower (e.g., about 1.5-3, 2-3, 2-4, 2-5, 2-10, 2-20, 2-50, 3-5, 3-10, 3-20, 3-50, 4-5, 4-10, 4-20, 4-50, 5-10, 5-15, 5-20, or 5-50-fold lower) than the reference expression or activation level of the corresponding analyte in a cell from non-inflamed and/or non-involved gastrointestinal tissue, in a cell from inflamed and/or involved gastrointestinal tissue, in a cell not treated with the therapeutic IBD drug, in a therapeutic IBD drug-sensitive cell treated with the therapeutic IBD drug, or in a therapeutic IBD drug-resistant cell treated with the therapeutic IBD drug. In some embodiments, a lower level of total expression or activation of the one or more analytes (e.g., epithelial cell analytes such as HER1, HER2, HER3, cMET, IGF-1R, PI3K, AKT, PRAS40, MEK, RSK, JAK1, STAT1, and STAT3) compared to a the reference (e.g., a sample from a control patient with IBD) indicates that the individual has inflamed and/or involved gastrointestinal tissue. In certain embodiments, a lower expression level of TNFα relative to the control indicates that the individual has non-inflamed and/or non-involved tissue. Furthermore, the individual may be undergoing or have undergone mucosal healing.

Non-limiting examples of analytes such as signal transduction molecules that can be interrogated for expression (e.g., total amount) levels and/or activation (e.g., phosphorylation) levels in a cell lysate include receptor tyrosine kinases, non-receptor tyrosine kinases, tyrosine kinase signaling cascade components, nuclear hormone receptors, nuclear receptor coactivators, nuclear receptor repressors, and combinations thereof.

In some embodiments, determining the expression level of the one or more analytes comprises detecting the total amount of each of the one or more analytes in the cell lysate with one or more antibodies specific for the corresponding analyte. In particular embodiments, the antibodies bind to the analyte irrespective of the activation state of the analyte to be detected, i.e., the antibodies detect both the non-activated and activated forms of the analyte.

Total expression level and/or status can be determined using any of a variety of techniques. In certain embodiments, the total expression level and/or status of each of the one or more analytes such as signal transduction molecules in a sample is detected with an immunoassay such as a single detection assay or a proximity dual detection assay (e.g., a Collaborative Enzyme Enhanced Reactive Immunoassay (CEER™)) as described herein.

In certain embodiments, determining the expression (e.g., total) levels of the one or more analytes comprises:
(i) incubating (e.g., contacting) a cell lysate produced from the cell with one or a plurality of dilution series of capture antibodies (e.g., capture antibodies specific for one or more analytes) to form a plurality of captured analytes, wherein the capture antibodies are restrained on a solid support (e.g., to transform the analytes present in the cell lysate into complexes of captured analytes comprising the analytes and capture antibodies);
(ii) incubating (e.g., contacting) the plurality of captured analytes with detection antibodies comprising one or a plurality of first and second activation state-independent antibodies specific for the corresponding analytes (e.g., first and second activation state-independent antibodies specific for the one or more analytes) to form a plurality of detectable captured analytes (e.g., to transform the complexes of captured analytes into complexes of detectable captured analytes comprising the captured analytes and detection antibodies),
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(iii) incubating (e.g., contacting) the plurality of detectable captured analytes with a second member of the signal amplification pair to generate an amplified signal; and
(iv) detecting the amplified signal generated from the first and second members of the signal amplification pair.

The first activation state-independent antibodies may be directly labeled with the facilitating moiety or indirectly labeled with the facilitating moiety, e.g., via hybridization between an oligonucleotide conjugated to the first activation state-independent antibodies and a complementary oligonucleotide conjugated to the facilitating moiety. Similarly, the second activation state-independent antibodies may be directly labeled with the first member of the signal amplification pair or indirectly labeled with the first member of the signal amplification pair, e.g., via binding between a first member of a binding pair conjugated to the second activation state-independent antibodies and a second member of the binding pair conjugated to the first member of the signal amplification pair. In certain instances, the first member of the binding pair is biotin and the second member of the binding pair is an avidin such as streptavidin or neutravidin.

In some embodiments, the facilitating moiety may be, for example, glucose oxidase. In certain instances, the glucose oxidase and the first activation state-independent antibodies can be conjugated to a sulfhydryl-activated dextran molecule as described in, e.g., U.S. Pat. No. 8,163,499. The sulfhydryl-activated dextran molecule typically has a molecular weight of about 500 kDa (e.g., about 250, 300, 350, 400, 450, 500, 550, 600, 650, 700, or 750 kDa). In other embodiments, the oxidizing agent may be, for example, hydrogen peroxide (H₂O₂). In yet other embodiments, the first member of the signal amplification pair may be, for example, a peroxidase such as horseradish peroxidase (HRP). In further embodiments, the second member of the signal amplification pair may be, for example, a tyramide reagent (e.g., biotin-tyramide). Preferably, the amplified signal is generated by peroxidase oxidization of biotin-tyramide to produce an activated tyramide (e.g., to transform the biotin-tyramide into an activated tyramide). The activated tyramide may be directly detected or indirectly detected, e.g., upon the addition of a signal-detecting reagent. Non-limiting examples of signal-detecting reagents include streptavidin-labeled fluorophores and combinations of streptavidin-labeled peroxidases and chromogenic reagents such as, e.g., 3,3′,5,5′-tetramethylbenzidine (TMB).

In certain instances, the horseradish peroxidase and the second activation state-independent antibodies can be conjugated to a sulfhydryl-activated dextran molecule. The sulfhydryl-activated dextran molecule typically has a molecular weight of about 70 kDa (e.g., about 40, 45, 50, 55, 60, 65, 70, 75, 80, 85, 90, 95, or 100 kDa).

In some embodiments, each dilution series of capture antibodies comprises a series of descending capture antibody concentrations. In certain instances, the capture antibodies are serially diluted at least 2-fold (e.g., 2, 5, 10, 20, 50, 100, 500, or 1000-fold) to produce a dilution series comprising a set number (e.g., 2, 3, 4, 5, 6, 7, 8, 9, 10, 15, 20, 25, or more) of descending capture antibody concentrations which are spotted onto an array. Preferably, at least 2, 3, 4, 5, or 6 replicates of each capture antibody dilution are spotted onto the array.

In other embodiments, the solid support comprises glass (e.g., a glass slide), plastic, chips, pins, filters, beads, paper, membrane (e.g., nylon, nitrocellulose, polyvinylidene fluoride (PVDF), etc.), fiber bundles, or any other suitable substrate. In a preferred embodiment, the capture antibodies are restrained (e.g., via covalent or noncovalent interactions) on glass slides coated with a nitrocellulose polymer such as, for example, FAST® Slides, which are commercially available from Whatman Inc. (Florham Park, N.J.). Exemplary methods for constructing antibody arrays suitable for use in the invention are described, e.g., in U.S. Pat. No. 8,163,499 and U.S. App. Publication No. US 2013/045880, the disclosures of which are herein incorporated by reference in their entirety for all purposes.

In further embodiments, determining the activation levels of the one or more analytes comprises detecting a phosphorylation level of the one or more analytes in the cell lysate with antibodies specific for the phosphorylated form of each of the analytes to be detected.

Phosphorylation levels and/or status can be determined using any of a variety of techniques. For example, it is well known in the art that phosphorylated proteins can be detected via immunoassays using antibodies that specifically recognize the phosphorylated form of the protein (see, e.g., Lin et al., Br. J. Cancer, 93:1372-1381 (2005)). Immunoassays generally include immunoblotting (e.g., Western blotting), RIA, and ELISA. More specific types of immunoassays include antigen capture/antigen competition, antibody capture/antigen competition, two-antibody sandwiches, antibody capture/antibody excess, and antibody capture/antigen excess. Methods of making antibodies are described herein and in Harlow and Lane, Antibodies: A Laboratory Manual, 1988, Cold Spring Harbor Laboratory Press, Cold Spring Harbor, N.Y., USA. Phospho-specific antibodies can be made de novo or obtained from commercial or noncommercial sources. Phosphorylation levels and/or status can also be determined by metabolically labeling cells with radioactive phosphate in the form of [γ-³²P]ATP or [γ-³³P]ATP. Phosphorylated proteins become radioactive and hence traceable and quantifiable through scintillation counting, radiography, and the like (see, e.g., Wang et al., J. Biol. Chem., 253:7605-7608 (1978)). For example, metabolically labeled proteins can be extracted from cells, separated by gel electrophoresis, transferred to a membrane, probed with an antibody specific for a particular analyte and subjected to autoradiography to detect ³²P or ³³P. Alternatively, the gel can be subjected to autoradiography prior to membrane transference and antibody probing.

In particular embodiments, the activation (e.g., phosphorylation) level and/or status of each of the one or more analytes in a sample is detected with an immunoassay such as a single detection assay or a proximity dual detection assay (e.g., a Collaborative Enzyme Enhanced Reactive Immunoassay (CEER™)) as described herein.

In other embodiments, the total expression level and/or activation level of the one or more analytes is expressed as “−”, “±”, “+”, “++”, “+++”, or “++++” that corresponds to increasing signal intensity for a particular analyte of interest that is determined using, e.g., a proximity assay such as CEER™. In some instances, an undetectable or minimally detectable level of total expression or activation of a particular analyte of interest that is determined using, e.g., a proximity assay such as CEER™, may be expressed as “−” or “±”. In other instances, a low level of total expression or activation of a particular analyte of interest that is determined using, e.g., a proximity assay such as CEER™, may be expressed as “+”. In yet other instances, a moderate level of total expression or activation of a particular analyte of interest that is determined using, e.g., a proximity assay such as CEER™, may be expressed as “++”. In still yet other instances, a high level of total expression or activation of a particular analyte of interest that is determined using, e.g., a proximity assay such as CEER™, may be expressed as “+++”. In further instances, a very high level of total expression or activation of a particular analyte of interest that is determined using, e.g., a proximity assay such as CEER™, may be expressed as “++++”.

In yet other embodiments, the expression level and/or activation level of the one or more analytes is quantitated by calibrating or normalizing the RFU value that is determined using, e.g., a proximity assay such as CEER™, against a standard curve generated for the particular analyte of interest. In certain instances, a computed units (CU) value can be calculated based upon the standard curve. In other instances, the CU value can be expressed as “−”, “±”, “+”, “++”, “+++”, or “++++” in accordance with the description above for signal intensity.

In certain embodiments, the total expression or activation level of a particular analyte of interest, when expressed as “−”, “±”, “+”, “++”, “+++”, or “++++”, may correspond to a level of expression or activation that is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or 100-fold higher or lower (e.g., about 1.5-3, 2-3, 2-4, 2-5, 2-10, 2-20, 2-50, 3-5, 3-10, 3-20, 3-50, 4-5, 4-10, 4-20, 4-50, 5-10, 5-15, 5-20, or 5-50-fold higher or lower) than a reference expression level or activation level, e.g., when compared to a negative control such as an IgG control, when compared to a standard curve generated for the analyte of interest, when compared to a positive control such as a pan-CK control, when compared to an expression or activation level determined in a cell from non-inflamed and/or non-involved gastrointestinal tissue, when compared to an expression or activation level determined in a cell from inflamed and/or involved gastrointestinal tissue, when compared to an expression or activation level determined in the presence of a therapeutic IBD drug, and/or when compared to an expression or activation level determined in the absence of an therapeutic IBD drug. In some instances, the correlation is analyte-specific. As a non-limiting example, a “+” level of expression or activation determined using, e.g., a proximity assay such as CEER™, may correspond to a 2-fold increase in expression or activation for one analyte and a 5-fold increase for another analyte when compared to a reference expression or activation level.

In some embodiments, the level of an therapeutic IBD drug such as an anti-TNFα drug is measured by size exclusion chromatography, e.g., size exclusion-high pressure chromatography (SE-HPLC), in the cell lysate of a tissue sample taken from an individual. Detailed descriptions of SE-HPLC are found in, for example, U.S. Pat. No. 8,574,855 and U.S. App. Publication No. 2013/0295685, the disclosures of which are herein incorporated by reference in their entirety for all purposes.

D. Production of Antibodies

The generation and selection of antibodies not already commercially available for analyzing the levels of total expression and activation of signal transduction molecules in gastrointestinal cells in accordance with the immunoassays of the present invention can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic (e.g., retain the functional binding regions of) antibodies can also be prepared from genetic information by various procedures. See, e.g., Antibody Engineering: A Practical Approach, Borrebaeck, Ed., Oxford University Press, Oxford (1995); and Huse et al., J. Immunol., 149:3914-3920 (1992).

Those skilled in the art will recognize that many approaches can be taken in producing antibodies or binding fragments and screening and selecting for affinity and specificity for the various polypeptides of interest, but these approaches do not change the scope of the present invention.

A more detailed description of polyclonal antibodies, monoclonal antibodies, humanized antibodies, human antibodies, bispecific antibodies, fragments thereof, and methods of purifying antibodies is found in U.S. App. Publication No. 2011/071042, the disclosure of which is herein incorporated by reference in its entirety for all purposes.

E. Statistical Analysis

In certain aspects, the present invention provides models to determine whether a subject having inflammatory bowel disease (IBD) has non-inflamed and/or non-involved or inflamed and/or involved gastrointestinal tissue. In other aspects, the present invention provides models to select a suitable or optimal IBD therapy for the treatment of a subject having IBD or to monitor the therapeutic response to IBD therapy in a subject having IBD. In further aspects, the present invention provides models to diagnose early onset IBD in a subject. In particular embodiments, the model is an algorithmic model which uses the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof.

An algorithmic model includes any of a variety of statistical methods and models used to determine relationships between variables. In the present invention, the variables are the values of the one or more analytes measured in accordance with the methods of the present invention. Any number of analytes can be analyzed using a statistical analysis described herein. For example, the value of 1, 2, 3, 4, 5, 6, 7, 8, 9, 10, 11, 12, 13, 14, 15, 16, 17, 18, 19, 20, 25, 30, 35, 40, 45, 50, 55, 60, or more analytes can be included in a statistical analysis such as, e.g., a quartile analysis or a multiple logistic regression model.

In particular embodiments, quantile analysis is applied to the value of one or more analytes to identify inflammation in gastrointestinal tissue or to guide treatment decisions for patients receiving IBD therapy. In other embodiments, one or a combination of two of more statistical algorithms such as learning statistical classifier systems are applied to the value of one or more analytes to identify inflammation in gastrointestinal tissue or to guide treatment decisions for patients receiving IBD therapy.

The algorithmic model includes the value of one or more analytes along with a statistical algorithm such as a quartile analysis or a multiple logistic regression analysis. In certain instances, the model has been trained with known outcomes using a training set cohort of samples. The algorithm is then validated using a validation cohort. Patient unknown samples can then be predicted based on the trained algorithms.

The term “statistical analysis” or “statistical algorithm” or “statistical process” includes any of a variety of statistical methods and models used to determine relationships between variables. In the present invention, the variables are the values or measurements of the one or more analytes described herein. Any number of analytes can be analyzed using a statistical analysis described herein. In some embodiments, logistic regression is used (e.g., a multiple logistic regression model). In other embodiments, linear regression is used. In further embodiments, a Cox proportional hazards regression model is used.

In certain embodiments, the statistical analysis of the present invention comprises a quantile measurement of one or more analytes within a given population. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of analyte levels (e.g., tertiles, quartile, quintiles, etc.), or their cumulative indices (e.g., quartile sums of analyte levels to obtain quartile sum scores (QSS), etc.) as variables in the statistical analyses (just as with continuous variables).

In particular embodiments, the statistical analysis comprises one or more learning statistical classifier systems. As used herein, the term “learning statistical classifier system” includes a machine learning algorithmic technique capable of adapting to complex data sets (e.g., panel of analytes) and making decisions based upon such data sets. In some embodiments, a single learning statistical classifier system such as a decision/classification tree (e.g., random forest (RF) or classification and regression tree (C&RT)) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and http://stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the C&RT software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM^{light} software developed by Thorsten Joachims (Cornell University) or using the LIBSVM software developed by Chih-Chung Chang and Chih-Jen Lin (National Taiwan University).

The various statistical methods and models described herein can be trained and tested using a cohort of samples (e.g., serological samples) from healthy individuals, patients with the disease or disorder of interest (e.g., IBD patients such as CD and/or UC patients), and/or patients on therapy (e.g., anti-TNFα drug therapy). For example, samples from patients diagnosed by a physician, and preferably by a gastroenterologist, as having IBD or a clinical subtype thereof using a biopsy, colonoscopy, or an immunoassay as described in, e.g., U.S. Pat. No. 6,218,129, are suitable for use in training and testing the statistical methods and models of the present invention. Samples from patients diagnosed with IBD can also be stratified into Crohn's disease or ulcerative colitis using an immunoassay as described in, e.g., U.S. Pat. Nos. 5,750,355 and 5,830,675. Samples from healthy individuals can include those that were not identified as IBD samples. One skilled in the art will know of additional techniques and diagnostic criteria for obtaining a cohort of patient samples that can be used in training and testing the statistical methods and models of the present invention.

The statistical methods and models described herein can be selected such that the sensitivity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The statistical methods and models described herein can be selected such that the specificity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The statistical methods and models described herein can be selected such that the negative predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The statistical methods and models described herein can be selected such that the positive predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The statistical methods and models described herein can be selected for a disease prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a clinician's office such as a gastroenterologist's office or a general practitioner's office.

The statistical methods and models described herein can be selected such that the overall accuracy is at least about 40%, and can be, e.g., at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In certain embodiments, the statistical analysis comprises calculating or applying a hazard ratio (HR). In certain instances, the HR is calculated using a Cox Proportional Hazard Model. The Cox regression model provides an estimate of the hazard ratio and its confidence interval. The confidence interval provides an estimate of the precision of the HR. A large confidence interval indicates a lower HR precision, while a small confidence interval indicates an HR with a high precision. A p-value indicates whether the HR is statistically significant. In some embodiments, the hazard is the development of inflamed and/or involved gastrointestinal tissue and the HR is the multiplicative effect on the hazard.

F. IBD Therapies and Methods of Administration

According to the methods of the present invention, the therapeutic IBD drugs described herein are administered to a subject by any convenient means known in the art. The methods of the present invention can be used to select a suitable therapeutic IBD drug or combination of therapeutic IBD drugs for the treatment of IBD in a subject. The methods of the present invention can also be used to identify the response of a gastrointestinal cell, e.g., a gastrointestinal cell of a subject with IBD, in a subject to treatment with a therapeutic IBD drug or combination of therapeutic IBD drugs. In addition, the methods of the present invention can be used to predict the response of a subject having an inflamed and/or involved or non-inflamed and/or non-involved gastrointestinal tissue to treatment with a therapeutic IBD drug or combination of therapeutic IBD drugs. One skilled in the art will appreciate that the therapeutic IBD drugs described herein can be administered alone or as part of a combined therapeutic approach with other types of therapies and/or surgery.

In certain embodiments, the therapeutic IBD drug comprises an anti-TNFα drug, an anti-signaling agent (i.e., a cytostatic drug) such as a monoclonal antibody or a tyrosine kinase inhibitor, a growth factor-driven epithelial signaling inhibitor (e.g., a HER1 inhibitor, a HER2 inhibitor, a HER3 inhibitor, a pan-HER inhibitor, a cMET inhibitor, a IGF-1R inhibitor), a JAK inhibitor, a PI3K inhibitor, an AKT inhibitor, an ERK inhibitor, a MEK inhibitor, an mTOR inhibitor, and/or any other compound with the ability to reduce or abrogate the inflammation of inflamed and/or involved gastrointestinal cells.

Non-limiting examples of anti-TNFα drugs include infliximab (REMICADE™, Johnson and Johnson), adalimumab (HUMIRATm, Abbott Laboratories), etanercept (ENBREL™ Amgen), certolizumab pegol (CIMZIA®, UCB, Inc.), golimumab (SIMPONI®; CNTO 148), CDP 571 (Celltech), and CDP 870 (Celltech), as well as other compounds which inhibit TNF-α activity.

Examples of anti-signaling agents include, without limitation, monoclonal antibodies such as trastuzumab (Herceptin®), pertuzumab (2C4), alemtuzumab (Campath®), bevacizumab (Avastin®), cetuximab (Erbitux®), gemtuzumab (Mylotarg®), panitumumab (Vectibix™) rituximab (Rituxan®), and tositumomab (BEXXAR®); tyrosine kinase inhibitors such as gefitinib (Iressa®), sunitinib (Sutent®), erlotinib (Tarceva®), lapatinib (GW-572016; Tykerb®), canertinib (CI 1033), semaxinib (SU5416), vatalanib (PTK787/ZK222584), sorafenib (BAY 43-9006; Nexavar®), imatinib mesylate (Gleevec®), leflunomide (SU101), vandetanib (ZACTIMA™; ZD6474), pelitinib, CP-654577, CP-724714, HKI-272, PKI-166, AEE788, BMS-599626, HKI-357, BIBW 2992, ARRY-334543, JNJ-26483327, and JNJ-26483327; and combinations thereof.

Non-limiting examples of pan-HER inhibitors include PF-00299804, neratinib (HKI-272), AC480 (BMS-599626), BMS-690154, PF-02341066, HM781-36B, CI-1033, BIBW-2992, and combinations thereof.

Non-limiting examples of HER2 inhibitors include monoclonal antibodies such as trastuzumab (Herceptin®) and pertuzumab (2C4); small molecule tyrosine kinase inhibitors such as gefitinib (Iressa®), erlotinib (Tarceva®), pelitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, JNJ-26483327, and JNJ-26483327; and combinations thereof.

Non-limiting examples of c-Met inhibitors include monoclonal antibodies such as AMG102 and MetMAb; small molecule inhibitors of c-Met such as ARQ197, JNJ-38877605, PF-04217903, SGX523, GSK 1363089/XL880, XL184, MGCD265, and MK-2461; and combinations thereof.

Exemplary ERK inhibitors for use in the present invention include, but are not limited to, Raf-1 inhibitors, such as sorafenib (Nexavar™, GlaxoSmithKline), dabrafenib (Tafinlar™, GlaxoSmithKline), XL281 (Exelixis), SB90885RAF265 (Novartis), GW5074, BAY 43-9006 (Bayer Healthcare Pharmaceuticals), and ISIS 5132 (ISIS Therapeutics); B-RAF inhibitor such as PLX4720 and vemurafenib (Zelboraf®, Genentech); MEK1/2 inhibitors, such as BAY86-9766 (Bayer Healthcare Pharmaceuticals), MEK162 (e.g., ARRY-162, Novartis), G-573 (Genentech), GDC-0623 (Genentech), GSK1120212 (GlaxoSmithKline), PD98059 (Pfizer), PD184352 (Pfizer), PD0325901 (Pfizer), U0126, AS703026/MSC1935369 (Merck), selumetinib (AZD6244/ARRY-142886, AstraZeneca) and trametinib (Mekinist™, GlaxoSmithKline); MAPK pathway inhibitors such as SCH772984 (Merck); and combinations thereof.

Exemplary mTOR inhibitors include, but are not limited to, sirolimus (rapamycin), temsirolimus (CCI-779), everolimus (RAD001), BEZ235, XL765, and combinations thereof.

Non-limiting examples of AKT inhibitors include 1L6-hydroxymethyl-chiro-inositol-2-(R)-2-O-methyl-3-O-octadecyl-sn-glycerocarbonate, 9-methoxy-2-methylellipticinium acetate, 1,3-dihydro-1-(1-((4-(6-phenyl-1H-imidazo[4,5-g]quinoxalin-7-yl)phenyl)methyl)-4-piperidinyl)-2H-benzimidazol-2-one, 10-(4′-(N-diethylamino)butyl)-2-chlorophenoxazine, 3-formylchromone thiosemicarbazone (Cu(II)Cl₂ complex), API-2, a 15-mer peptide derived from amino acids 10-24 of the proto-oncogene TCL1 (Hiromura et al., J. Biol. Chem., 279:53407-53418 (2004), KP372-1, the compounds described in Kozikowski et al., J. Am. Chem. Soc., 125:1144-1145 (2003) and Kau et al., Cancer Cell, 4:463-476 (2003), and combinations thereof.

Non-limiting examples of PI3K inhibitors include PX-866, wortmannin, LY 294002, quercetin, tetrodotoxin citrate, thioperamide maleate, GDC-0941 (957054-30-7), IC87114, PI-103, PIK93, BEZ235 (NVP-BEZ235), TGX-115, ZSTK474, (−)-deguelin, NU 7026, myricetin, tandutinib, GDC-0941 bismesylate, GSK690693, KU-55933, MK-2206, OSU-03012, perifosine, triciribine, XL-147, PIK75, TGX-221, NU 7441, PI 828, XL-765, WHI-P 154, and combinations thereof.

Non-limiting examples of MEK inhibitors include PD98059, ARRY-162, RDEA119, U0126, GDC-0973, PD184161, AZD6244, AZD8330, PD0325901, ARRY-142886, and combinations thereof.

Therapeutic IBD drugs can be administered with a suitable pharmaceutical excipient as necessary and can be carried out via any of the accepted modes of administration. Thus, administration can be, for example, oral, buccal, sublingual, gingival, palatal, intravenous, topical, subcutaneous, transcutaneous, transdermal, intramuscular, intra joint, parenteral, intra-arteriole, intradermal, intraventricular, intracranial, intraperitoneal, intravesical, intrathecal, intralesional, intranasal, rectal, vaginal, or by inhalation. By “co-administer” it is meant that a therapeutic IBD drug is administered at the same time, just prior to, or just after the administration of a second drug (e.g., another therapeutic IBD drug, a drug useful for reducing the side-effects associated with therapeutic IBD drug therapy, an immunotherapeutic agent, etc.).

A therapeutically effective amount of a therapeutic IBD drug may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for human subjects and other mammals, each unit containing a predetermined quantity of a therapeutic IBD drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic IBD drug.

Methods for preparing such dosage forms are known to those skilled in the art (see, e.g., REMINGTON'S PHARMACEUTICAL SCIENCES, 18TH ED., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with a therapeutic IBD drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. A therapeutic IBD drug can also be formulated into a suppository disposed, for example, in a polyethylene glycol (PEG) carrier.

Liquid dosage forms can be prepared by dissolving or dispersing a therapeutic IBD drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. A therapeutic IBD drug can also be formulated into a retention enema.

For topical administration, the therapeutically effective dose can be in the form of emulsions, lotions, gels, foams, creams, jellies, solutions, suspensions, ointments, and transdermal patches. For administration by inhalation, a therapeutic IBD drug can be delivered as a dry powder or in liquid form via a nebulizer. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to a subject.

A subject can also be monitored at periodic time intervals to assess the efficacy of a certain therapeutic regimen. For example, the total expression and/or activation levels of certain signal transduction molecules may change based on the therapeutic effect of treatment with one or more of the therapeutic IBD drugs described herein. The subject can be monitored to assess response and understand the effects of certain drugs or treatments in an individualized approach. Additionally, subjects who initially respond to a specific therapeutic IBD drug or combination of therapeutic IBD drugs may become refractory to the drug or drug combination, indicating that these subjects have developed acquired drug resistance. These subjects can be discontinued on their current therapy and an alternative treatment prescribed in accordance with the methods of the present invention.

### IV. Examples

The following examples are offered to illustrate, but not to limit, the claimed invention.

### Example 1

### Signaling Pathway Proteins are Associated with Inflammation and Tissue Location in IBD Patient Tissues: Implications for Mucosal Healing

This example illustrates a method for determining whether a subject has inflammatory bowel disease and/or inflamed, involved, non-inflamed or non-involved gastrointestinal tissue, as well as whether the subject is undergoing or has undergone mucosal healing. This example also illustrates a method for selecting an optimal IBD therapy for the treatment of IBD in a subject. Furthermore, this example illustrates a method for monitoring the therapeutic response to IBD therapy in a subject having IBD including Crohn's disease and ulcerative colitis.

Endoscopic mucosal healing correlates with clinical outcomes and has been proposed as a goal for targeted therapeutics in inflammatory bowel disease (IBD). Moreover, it is a key prognostic factor in the management of IBD. However, very little is known about the specific molecular pathways that may be contributing to this process and the identity of biomarkers for mucosal healing. The goal of this study was to evaluate the presence and activity of growth factor- and cytokine-driven signaling pathway proteins in IBD tissue.

The study population consisted of 42 IBD patients from the Anti-TNF Tissue Level and Antibodies in Serum (ATLAS) study on maintenance anti-TNF therapy who underwent endoscopy for tissue collection. Two tissue samples were collected from each patient. For each sample location (colon or ileum), inflammation status based on endoscopic assessment and involvement in disease was recorded. Each sample was classified into 3 groups: involved tissue (overt inflammation), previously involved tissue (area had active disease in previous examinations, but showed no inflammation at the time) and non-involved tissue (no current or previous evidence of inflammation). Total and phosphorylated (activated) signal transduction proteins were measured in each frozen tissue sample using a Collaborative Enzyme Enhanced Reactive Immunoassay, CEER™ (Prometheus Laboratories, San Diego). These signal transduction proteins included total and activated (e.g., phosphorylated) HER1, HER2, HER3, cMET, IGF1R and PI3K, and phosphorylated AKT, PRAS40, MEK, RSK, STAT1, STAT3, and JAK1 proteins.

For statistical analysis, involved and previously involved samples were grouped and compared to non-involved samples using pairs of samples from twelve individuals, where each individual had one sample in each category. Similarly, pairs of non-involved tissues from six individuals were used for a comparison of colonic vs ileal tissue. Significance tests were paired Mann-Whitney U tests where p<0.05 was considered significant.

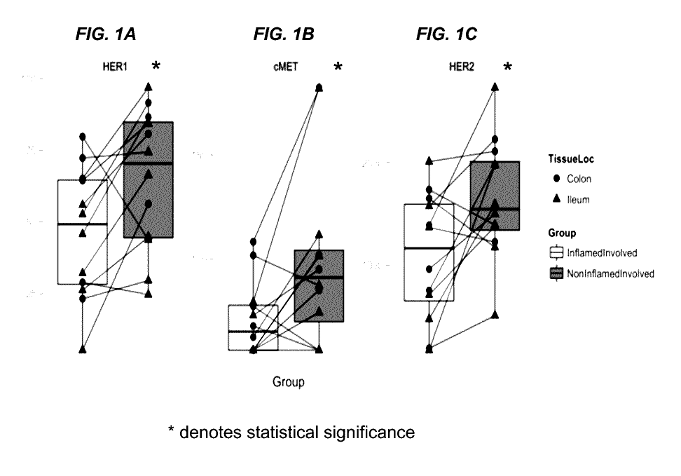

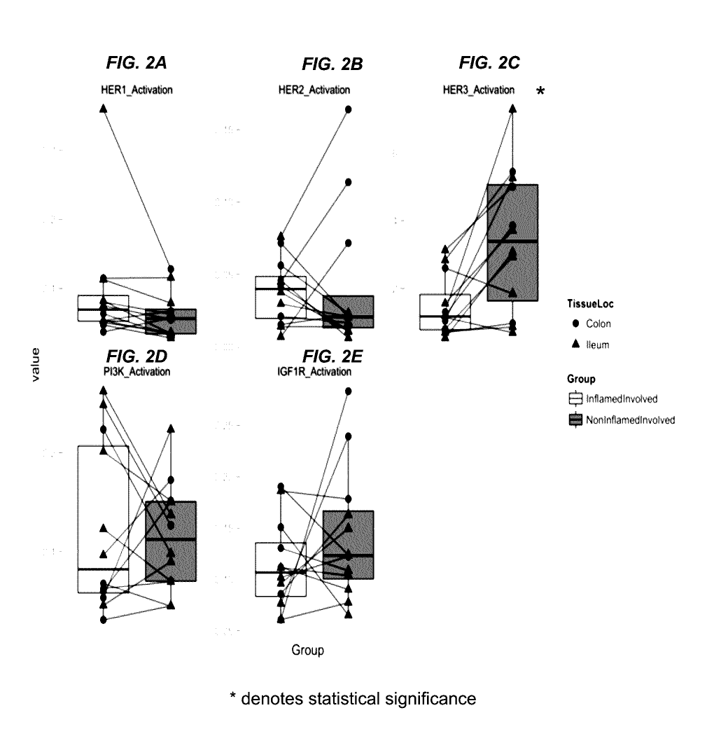

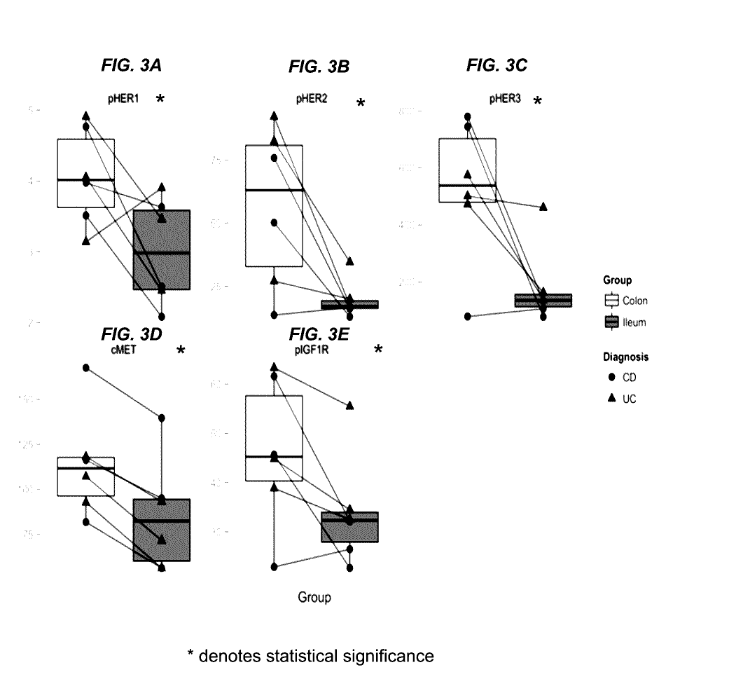

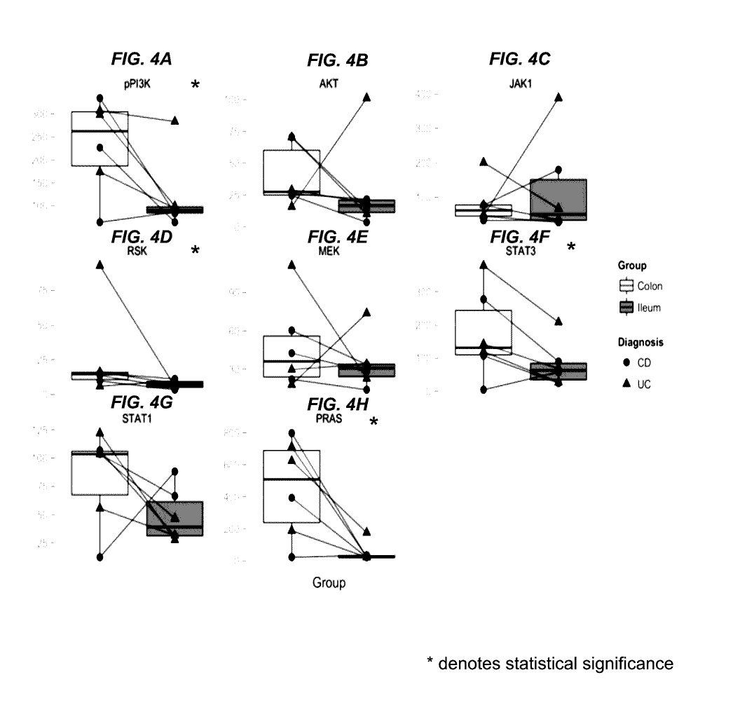

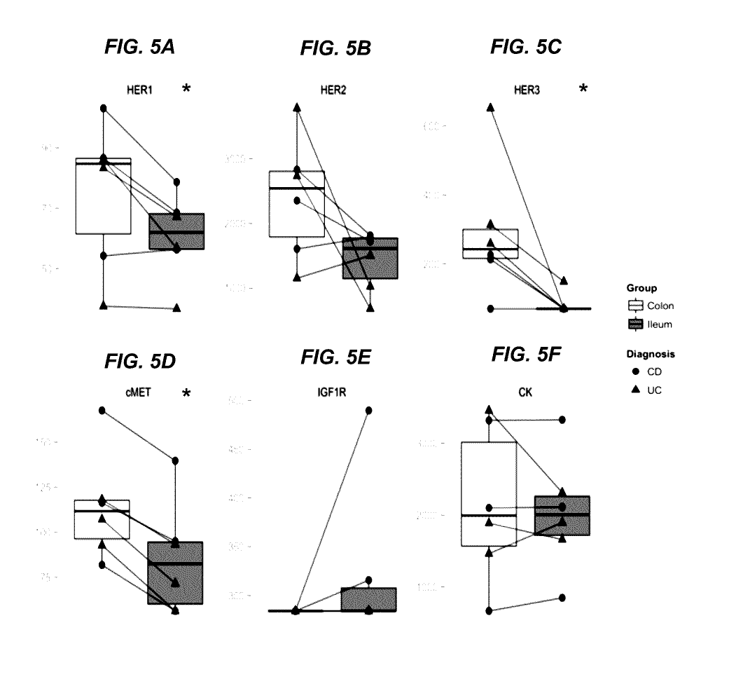

Total expression levels of growth factor driven signaling molecules such as HER1, HER2 and cMET was significantly lower in involved/previously involved tissues as compared to the non-inflamed tissues (see, Table 1 and FIGS. 1A-C), whereas the activation of these molecules was not significantly different in these tissues (FIGS. 2A-E). There were significant differences in signal transduction pathways between the colon and ileum in non-inflamed tissue (Table 2). A comparison of the colon vs. ileum in non-inflamed tissue demonstrated significant signal transduction differences. Colonic tissues demonstrated higher activation than ileal tissues for both growth factor driven signaling molecules (e.g., phosphorylated HER1, phosphorylated HER2, phosphorylated HER3 and phosphorylated cMET, and phosphorylated IGF-1R; see, Table 2, FIGS. 3A-E) and cytokine driven (JAK-STAT3) signaling molecules (see, Table 2, FIGS. 4A-H). Total cMET levels were higher in the colonic tissues vs. the ileal tissues (FIG. 3D). Both the MEK-RSK signaling module (FIGS. 4D and 4E), which is activated downstream of the growth factor driven receptors (HER1, HER2, HER3, cMET and IGF-1R), and the PI3K-AKT-PRAS40 signaling module, which is activated downstream of the growth factor receptors but also cross-talks with the JAK-STAT3 signaling pathway, were also higher in the colonic tissues than ileal tissues (see, Table 2 and FIGS. 4A-C and 4F-H). The total levels of several epithelial cell markers (e.g., analytes) were also significantly higher in non-inflamed tissue of the colon than of the ileum (see, FIG. 5). In particular, HER1 (FIG. 5A), HER3 (FIG. 5C) and cMET (FIG. 5D) levels were statistically significantly higher in colonic tissue vs. ileal tissue. The level of the reference (control) analyte, e.g., CK was not different between the colonic and ileal tissues (FIG. 5F).

TABLE 1
Total Levels of Growth Factor Driven
Signaling Molecules (e.g., Analytes)

| | Median Inflamed | Median Non- | |
|---|---|---|---|
| Marker | or Involved | Inflamed | p-value |
| HER1 | 49.2 | 70.4 | 0.01 |
| HER2 | 1165 | 1548 | 0.01 |
| cMET | 65.3 | 91.3 | 0.01 |

TABLE 2
Activated Levels of Signal Transduction Molecules (e.g., Analytes)

| Marker (CU/μg or | | | |
|---|---|---|---|
| *pg/μg) | | | |
| (“phos” refers to | Median Non- | Median Non- | |
| phosphorylated) | Inflamed Colon | Inflamed Ileum | p-value |
| Phos-HER1 | 4.0 | 3.0 | 0.047 |
| Phos-HER2 | 63 | 17 | 0.03 |
| Phos-HER3 | 537 | 135 | 0.03 |
| Phos-IGF1R | 45 | 32 | 0.03 |
| Phos-STAT3 | 131 | 63 | 0.03 |
| Phos-MEK* | 36 | 31 | 0.2 |
| Phos-RSK* | 15 | 7 | 0.03 |
| Phos-PI3K | 262 | 90 | 0.03 |
| Phos-AKT* | 27 | 16 | 0.2 |
| Phos-PRAS40 | 509 | 23 | 0.03 |

There were significant differences in signaling molecules in both the involved/previously involved vs. non-involved tissue, and in the non-involved colonic vs. ileal tissue. Lower levels of growth factor driven epithelial signaling molecules in involved IBD tissues are indicative of inflammation-mediated tissue injury, leading to a decrease in epithelial cells in involved tissue. Higher levels of activated growth factor and cytokine driven signaling molecules in the colon than in the ileum may be due to differences in the microbiome, pH or turnover of these tissues and may also reflect differences in immune-mediated signaling. The results illustrate the biological mechanism of mucosal healing in IBD patients and provide predictive, diagnostic, prognostic and/or pharmacodynamic markers for mucosal healing.

### Example 2

### Signaling Pathway Proteins are Associated with Inflammation and Disease Location in IBD: Implications for Mucosal Healing and Malignancy Risk

### Background

Little is known about the molecular pathways activated in the intestinal tissue of patients with inflammatory bowel diseases (IBD). This study aimed to evaluate the presence and activity of growth factor- and cytokine-driven signaling pathway proteins in intestinal tissue of patients with IBD.

### Methods

The study included 42 IBD patients seen at the Crohn's and Colitis center of the University if Miami (Florida, USA).

All patients underwent colonoscopy and had tissue samples collected from two anatomic sites. For each sample location (colon or ileum), inflammation status based on endoscopic assessment was classified into 3 groups:
A. Involved tissue (overt inflammation).
B. Previously involved tissue (area had overt active disease in previous examinations, but there was no evidence of disease activity when the samples were taken).
C. Non-involved tissue (no current or previous evidence of mucosal inflammation).

Total and phosphorylated (i.e., activated) signal transduction pathway proteins were measured in frozen tissue samples using a Collaborative Enzyme Enhanced Reactive (CEER) assay.

For statistical analysis, involved and previously involved samples (inflamed samples) were grouped and compared to non-involved samples (non-inflamed samples) using pairs of sections from twelve individuals, where each individual had one sample in each category. Similarly, pairs of non-involved tissues from six individuals were used for a comparison of colonic vs. ileal tissue.

### Results

Total expression of growth factor driven signaling molecules, HER1, HER2 and cMET, was significantly lower in inflamed IBD tissues as compared to the non-inflamed tissues. However, no differences were observed in the activation state of these molecules.

There were significant differences in the expression of signal transduction molecules between the colon and ileum in non-inflamed tissues. Colonic tissues demonstrated higher activation than ileal tissues for both growth factor driven (phosphorylated HER1, HER2, HER3, and IGF1R) and cytokine driven (STAT3) signaling molecules.

Both the MEK-RSK module, which is activated downstream of the growth factor driven receptors, and the PI3K-AKT-PRAS40 module, which is activated by growth factors but also cross-talks with the STAT3 signaling pathway, were also higher in the colonic tissues than ileal tissues. Significant differences were observed in the TNF/HER2 ratio and Drug/HER2 ratio between inflamed and non-inflamed tissues.

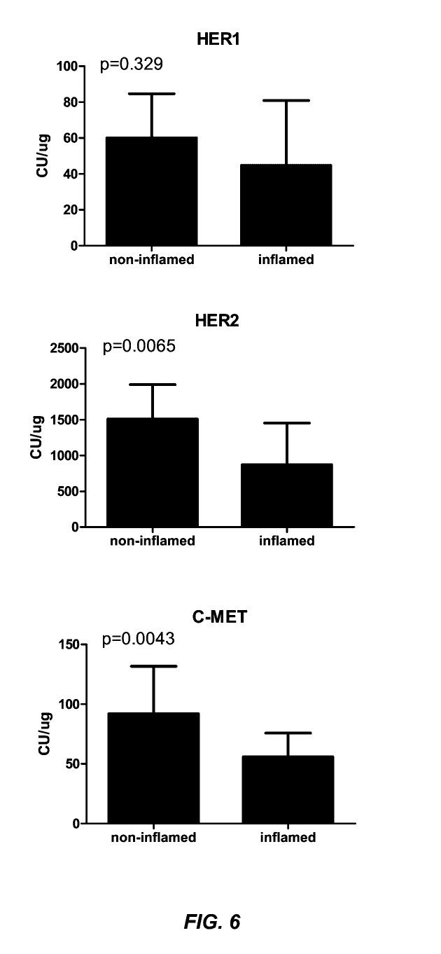

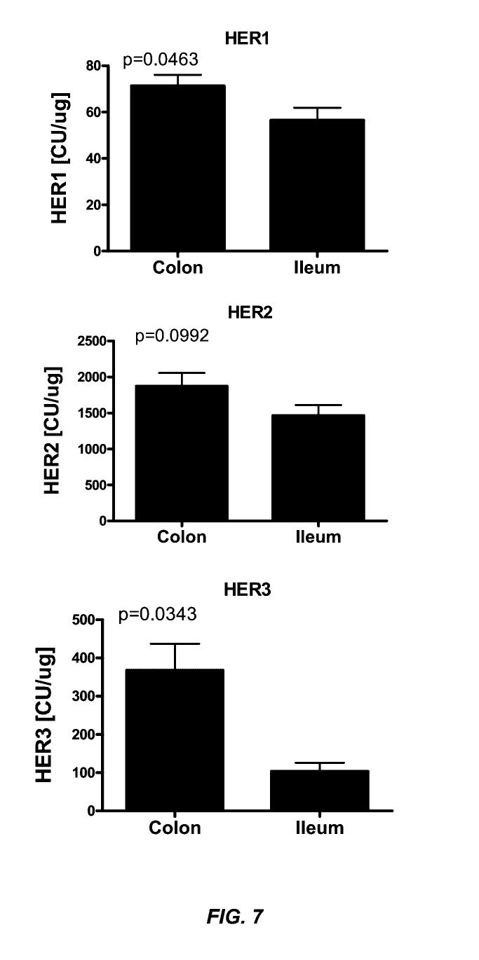

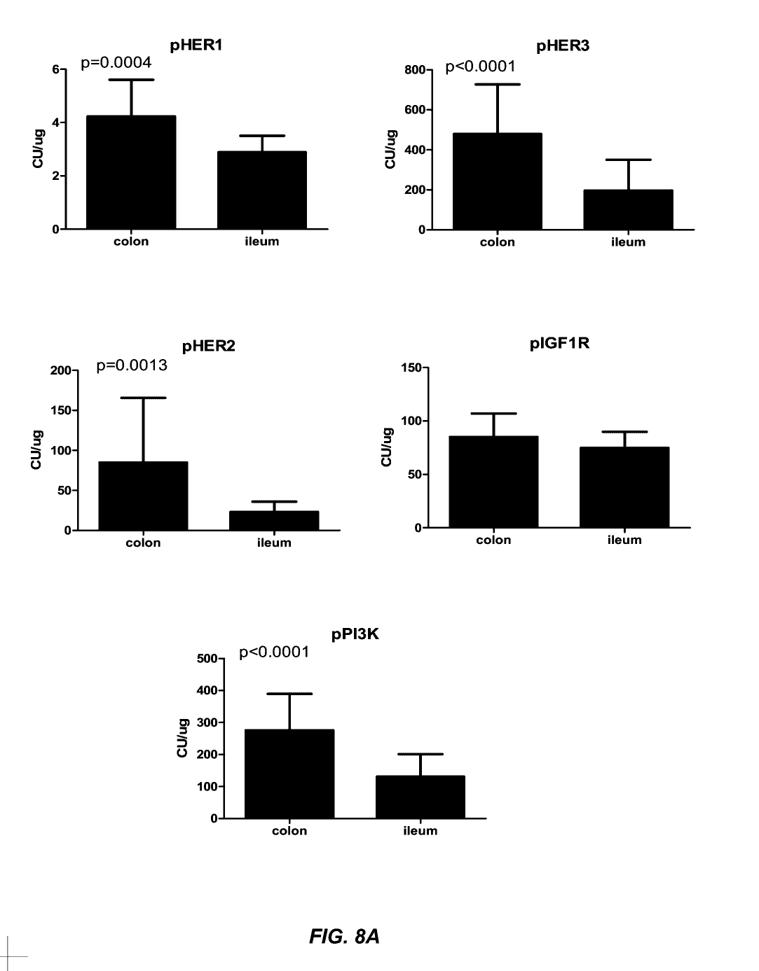

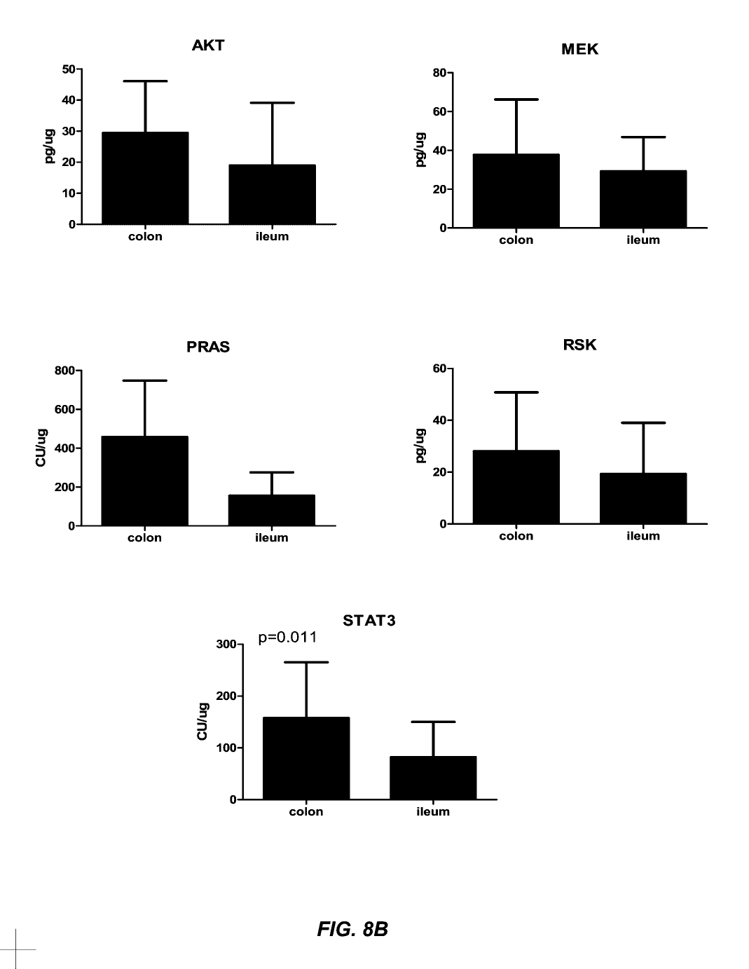

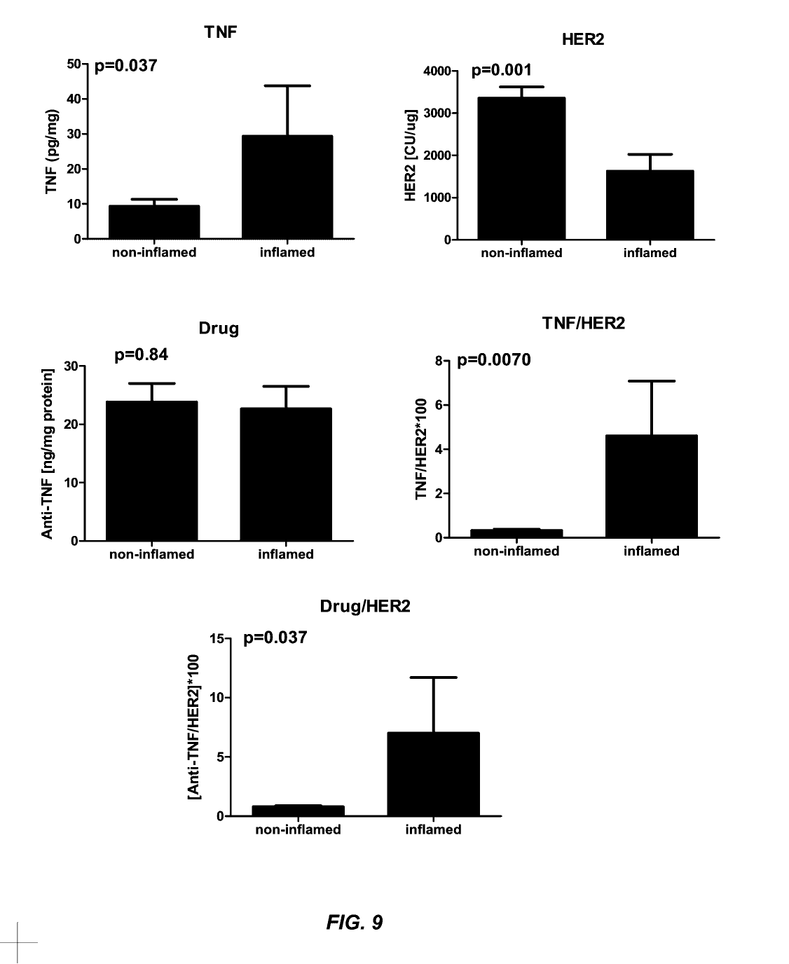

FIG. 6 shows that epithelial cell markers are higher in non-inflamed, non-involved IBD tissues than in inflamed or involved tissues. FIG. 7 shows that several epithelial cell markers are also significantly higher in the colon than in the ileum. FIGS. 8A-B show that growth factor and cytokine driven, activated signaling pathways are expressed to higher levels in the colon vs. the ileum. FIG. 9 shows that HER2 normalized TNF expression and Drug (i.e., anti-TNF drug) are significantly higher in inflamed IBD tissues as compared to non-inflamed tissues.

### CONCLUSIONS

Growth factor driven receptor tyrosine kinases and STAT signaling molecules are expressed in the intestinal epithelium of both IBD patients and unaffected individuals.

Inflamed tissues demonstrate a lower expression of signaling pathway molecules presumably due to massive bacterial and leukocyte infiltration and lower epithelial content (Swidsinski J. Phys and Pharm, 60:61-71).

Normalization to tissue epithelial content reveals that inflamed IBD tissues express higher levels of TNF with a higher concentration of anti-TNFs as compared to non-inflamed tissues.

Signaling pathway differences are also observed in the ileum vs. the colon with lower expression in the former.

Inherent differences in the signaling pathway expressions between the ileum and colon may represent differences in cancer risk between the two organs.

Measurement of signaling proteins in intestinal tissue may be useful for assessment of the extent of injured mucosa, prediction of dysplasia risk and optimization of anti-TNFs for mucosal healing.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for determining whether a subject having inflammatory bowel disease (IBD) has non-inflamed and/or non-involved or inflamed and/or involved gastrointestinal tissue, the method comprising:
(a) measuring the total level and/or activation level of one or more analytes selected from the group consisting of HER1, HER2, HER3, cMET, IGF-1R, PI3K, AKT, PRAS40, MEK, RSK, JAK1, STAT1, STAT3, TNFα, anti-TNFα drug, and a combination thereof in a cell lysate, wherein the cell lysate is produced by lysing a cell from a gastrointestinal tissue sample taken from the subject; and
(b) comparing the total level and/or activation level of the one or more analytes measured in step (a) to that of a control, thereby determining whether the subject has non-inflamed and/or non-involved or inflamed and/or involved gastrointestinal tissue.

2. The method of claim 1, wherein the total level of one or more analytes selected from the group consisting of HER1, HER2, cMET, and a combination thereof in the cell lysate is higher than that of the control, thereby determining that the subject has non-inflamed and/or non-involved gastrointestinal tissue.

3. The method of claim 1, wherein the activation level of HER3 in the cell lysate is higher than that of the control, thereby determining that the subject has non-inflamed and/or non-involved gastrointestinal tissue.

4. The method of claim 1, wherein the control is an inflamed and/or involved gastrointestinal tissue.

5. The method of claim 1, wherein the total level of one or more analytes selected from the group consisting of HER1, HER2, cMET, and a combination thereof in the cell lysate is lower than that of the control, thereby determining that the subject has inflamed and/or involved gastrointestinal tissue.

6. The method of claim 1, wherein the activation level of HER3 in the cell lysate is lower than that of the control, thereby determining that the subject has inflamed and/or involved gastrointestinal tissue.

7. The method of claim 1, wherein a ratio of the total level of TNFα to the total level of HER2 in the cell lysate is higher than that of the control and/or a ratio of the total level of the anti-TNFα drug to the total level of HER2 in the cell lysate is higher than that of the control, thereby determining that the subject has inflamed and/or involved gastrointestinal tissue.

8. The method of claim 1, wherein the control is a non-inflamed and/or non-involved gastrointestinal tissue.

9. The method of claim 1, wherein the gastrointestinal tissue sample is isolated from a subject using endoscopic ultrasound and fine needle aspiration.

10. The method of claim 1, wherein step (a) comprises measuring the total level and/or activation level of any combination of two, three, four, five, six, seven, eight, nine, ten, eleven, twelve, thirteen, fourteen, or fifteen of the analytes.

11. The method of claim 1, wherein the gastrointestinal tissue sample is isolated from a subject receiving IBD therapy.

12. The method of claim 11, wherein the IBD therapy is an anti-TNFα drug.

13. The method of claim 1, further comprising selecting a suitable IBD therapy based upon the total level and/or activation level of one or more analytes measured in step (a).

14. The method of claim 13, wherein the suitable IBD therapy comprises an anti-TNFα drug if the total level and/or activation level of one or more of analytes selected from the group consisting of HER1, HER2, HER3, cMET, IGF-1R, and a combination thereof is lower than that of the control.

15. The method of claim 13, wherein the suitable IBD therapy comprises a growth factor-driven epithelial signaling inhibitor drug if the total level and/or activation level of one or more of analytes selected from the group consisting HER1, HER2, HER3, cMET, IGF-1R, and a combination thereof is higher than that of the control.

16. The method of claim 13, wherein the suitable IBD therapy comprises a JAK inhibitor drug if the activation level of STAT3 is higher than that of the control.

17. The method of claim 13, wherein the suitable IBD therapy is selected from the group consisting of a PI3K inhibitor drug, an AKT inhibitor drug, an ERK inhibitor drug, a MEK inhibitor drug, an mTOR inhibitor drug, and a combination thereof if the activation level of one or more of analytes selected from the group consisting PI3K, AKT, PRAS40, MEK, RSK, and a combination thereof is higher than that of the control.

18. The method of claim 15, wherein the gastrointestinal tissue sample is taken from the colon of the subject.

19. The method of claim 15, wherein the control is a non-inflamed and/or non-involved ileal tissue.

20. The method of claim 1, wherein a transition from an inflamed and/or involved gastrointestinal tissue determined at an earlier time point to a non-inflamed and/or non-involved gastrointestinal tissue determined in step (b) indicates that the subject is undergoing or has undergone mucosal healing.
